(19) United States
(12) Patent Application Publication
(10) Pub. No.: US 2019/0142677 A1
(43) Pub. Date: May 16, 2019

(54) CLOUD-BASED OPHTHALMIC EYELID TREATMENT MONITORING SYSTEM AND METHODS OF THE SAME

(71) Applicant: EYEDETEC MEDICAL, INC., Danville, CA (US)

(72) Inventors: Barry J. LINDER, Danville, CA (US); Ronald D. LINDER, Gilbert, AZ (US); James L. DIPPO, Arvada, CO (US); Rubin D. LINDER, Danville, CA (US); John A. SCHOLL, San Ramon, CA (US)

(73) Assignee: EYEDETEC MEDICAL, INC., Danville, CA (US)

(21) Appl. No.: 16/244,820

(22) Filed: Jan. 10, 2019

Related U.S. Application Data
(63) Continuation-in-part of application No. 16/146,396, filed on Sep. 28, 2018.
Application No. 16/146,396 is a continuation-in-part of application No. 15/187,457, filed on Jun. 20, 2016.
(60) Provisional application No. 62/565,818, filed on Sep. 29, 2017.
(60) Provisional application No. 62/230,843, filed on Jun. 18, 2015.
(60) Provisional application No. 62/615,741, filed on Jan. 10, 2018.

(51) Int. Cl. A61H 1/00; H04L 29/08; A61F 7/03; A61B 5/00

(52) U.S. Cl. CPC A61H 1/00; H04L 67/10; A61F 2007/0004; A61B 5/4833; A61F 7/034

(57) ABSTRACT

Embodiments provide cloud-based ophthalmic eyelid treatment monitoring systems and cloud-based methods for monitoring ophthalmic eyelid treatment. The monitoring system includes a moldable warming device, a monitor, and a cloud server. The moldable warming device includes a heating disc, a resonance frequency stimulation vibration generator (RFSVG), a coupling device, a mask, and a sensor array. The mask is configured to hold the heating disc, the RFSVG, and the coupling device for use in parallel utility. The sensor array is configured to generate a data stream responsive to a vibration and heating profile of a user's individual patient eyelid and periorbital three-dimensional anatomy and surface topography. The monitor is configured to receive the data stream from the sensor array and configured to transmit the data stream to the cloud server. The cloud server is configured to process the data stream so as to determine tuning parameters of the vibration and heating profile of the user's individual patient eyelid and periorbital three-dimensional anatomy and surface topography, and configured to transmit the tuning parameters to the monitor. The monitor is configured to receive the tuning parameters from the cloud server, configured to display information related to the tuning parameters, and configured to transmit the tuning parameters to the moldable warming device. The moldable warming device is configured to receive the tuning parameters from the monitor and configured to generate thermal and vibratory energy according to the tuning parameters.

### CROSS REFERENCE TO RELATED APPLICATIONS

This application is a continuation-in-part application of U.S. patent application Ser. No. 15/187,457, filed on Jun. 20, 2016, entitled “MOLDABLE HEATER WITH MINIATURE HARMONIC RESONANCE FREQUENCY VIBRATION GENERATOR FOR OPHTHALMIC EYELID THERAPY,” which claims priority to U.S. Provisional Patent Application Ser. No. 62/230,843, filed on Jun. 18, 2015; this application is a continuation-in-part application of U.S. patent application Ser. No. 16/146,396, filed on Sep. 28, 2018, entitled “MOLDABLE HEATER WITH MINIATURE HARMONIC RESONANCE FREQUENCY VIBRATION GENERATOR FOR OPHTHALMIC EYELID THERAPY,” which claims priority to U.S. Provisional Patent Application Ser. No. 62/565,818, filed on Sep. 29, 2017; U.S. patent application Ser. No. 16/146,396 is a continuation-in-part application of the aforementioned U.S. patent application Ser. No. 15/187,457, filed on Jun. 20, 2016, which claims priority to the aforementioned U.S. Provisional Patent Application Ser. No. 62/230,843, filed on Jun. 18, 2015; this application claims priority to U.S. Provisional Application Ser. No. 62/615,741, filed on Jan. 10, 2018; all of the above-referenced applications are hereby incorporated by reference in their entireties into this application.

### BACKGROUND

### 1. Field

Embodiments relate generally to systems of and methods for treating and monitoring dry eye disease. More particularly, embodiments of the present disclosure relate to wearable eye masks with embedded sensors, cloud-based ophthalmic eyelid treatment monitoring systems, and cloud-based methods for monitoring ophthalmic eyelid treatment.

### 2. Description of the Related Art

Eye patch devices are known in the art. For example, U.S. Pat. No. 4,682,371 describes a protective eye patch. The '371 patch has several tabs for securing the patch to a patient's eye. U.S. Pat. No. 3,068,863 describes a patch designed to keep the eye closed. U.S. Pat. No. 3,092,103 describes a patch with a cushion material at the edge that allows the patient's eye to move underneath the eye patch. U.S. Pat. No. 3,908,645 describes an ophthalmic therapeutic pressure bandage with a conformable, permeable carrier tape.

U.S. Pat. No. 6,409,746 describes an eye pillow that releases steam from its surface applied to the eyes and the area around the eyes. The temperature described in the '746 patent is 50° C. or lower and has a total weight of 50 g or more.

Several conditions exist for which medical and cosmetic therapy is appropriate. For example, blepharitis, meibomitis, chalazia, and/or styes are common disorders of the eyelids that cause chronic inflammation in the peri-orbita, and are often associated with ocular tear film abnormalities resulting in dry eye disease and symptoms. Symptoms of dry eye disease and blepharitis include burning, itching, light sensitivity, blurred vision, tearing, and foreign body sensation. Signs include eyelash crusting, ocular discharge, eyelid scaling and swelling, corneal staining, and conjunctival redness. For example, staphylcoccal blepharatis is often associated with scaling and crusting along the eye lashes. There is no cure for dry eyes, and long-term treatment is required to keep it under control.

The predominant cause of dry eye is an insufficient or abnormal lipid layer of the surface of the tear film. In a healthy eye, this oily layer inhibits the evaporation of the water based sub layers of the tear film, thereby maintaining a stable tear film. These lipids are produced in the Meibomian glands located in the eyelids. From about 24 to about 40 Meibomian glands exist in each eyelid. For those suffering evaporative dry eye disease, the likely root cause is Meibomian glands that have become filled with viscous lipids, and occasionally clogged, resulting in a reduced quantity and abnormal quality of lipids flowing out onto the tear film. Meibomitis, also known as Meibomian Gland Dysfunction (MGD), is a dysfunction of the Meibomian gland and limits the gland's ability to provide a normal lipid-based oily layer as a critical component of the eye's natural tear film.

Currently available treatments for dry eye disease and related conditions include warm compresses for 5-15 minutes, such as a warm washcloth, that heats the debris and crust on the lid, and lowers the viscosity of the lipids in the Meibomian glands. After the lid has been warmed, occasionally a lid scrub is performed by using a suitable soap, such as Neutrogena® or Johnson's Baby Shampoo®. Commercially available cleansing pads are available to assist in performing the lid scrub, for example OCuSOFT® Lid Scrubs or Novartis Ophthalmics Eye Scrub®. Following the eye scrub, antibiotics, such as polysporin, tobramycin, or erythromycin can be applied, to alleviate patient discomfort and reinforce the treatment.

Warm moist compress therapy applied to the skin of the closed eyelids increases tear-film lipid layer thickness for subjects with MGD by more than 80% after 5 minutes of initiating treatment and an additional 20% after 15 minutes of treatment. The transition temperature from a solid to a liquid for Meibomian lipids is actually a range from 28° C. to 32° C. because of differences between individual's mixture of lipids. The temperature of the eyelids will therefore affect the liquidity of Meibomian lipids and hence their viscosity. The non-Newtonian lipid mixture is known to undergo shear thinning when exposed to shear forces. Further it is known that oscillations enhance the flow rate of a shear-thinning fluid.

Conventional ocular heating devices, such as warm compresses, typically require an external power source. These sources include electricity, a stove top boiling preparation, or a microwave appliance, and are consequently difficult to provide a controlled temperature to the eyelids, are labor intensive, cumbersome, and inconvenient, and therefore historically result in poor patient compliance and persistence with the recommended therapy. Some success is realized with in-office, doctor-assisted visits.

What is needed is a convenient, accurate, and effective, easily used hand moldable heating source that patients or their doctors apply via a coupling mechanism to patient's eyelids, and which delivers a therapeutic temperature to the entire eyelid surface independent of the individual's orbital anatomy, for a sufficient length of time to be effective. There is also a need for a device or component of the system that incorporates a moldable material to serve as a coupling element, heated and able to deliver heat and resonance frequency stimulation vibration to the target tissue of the eyelid, as well as to detect a positive eyelid resonance response from a broad range of generated harmonic frequencies, thus allowing a personalized or custom approach to each individual user.

Additionally, what is needed is a treatment monitoring system for physicians to provide the patient optimal thermal and resonance frequency stimulation vibratory profiles when using the moldable warming device. The personalized and optimized thermal and resonance frequency stimulation vibratory stimulation profiles may be obtained by utilizing wearable masks with embedded sensors that communicate with local or cloud computing technologies. There is also a need to provide physicians remote access to the thermal and vibratory profiles not only when the patient is in the physician's office being treated but also when the patient is at home conducting maintenance treatment.

### SUMMARY

Embodiments of the present disclosure relate generally to systems of and methods for treating and monitoring dry eye disease. More particularly, embodiments of the present disclosure relate to wearable masks with embedded sensors that communicate with cloud-based ophthalmic eyelid treatment monitoring systems and cloud-based methods for monitoring ophthalmic eyelid treatment.

Embodiments relate to a reusable system and one-time use or reusable components for a heating device with a reusable miniature harmonic frequency stimulation generator inside a reusable eye mask with a reusable or one-time use coupling device. The system according to various embodiments ensures proper delivery of targeted heat therapy and appropriately tuned harmonic resonance frequency stimulation to the location of the eyelids, from the inner surface of the eyelid or over their entire eyelid external skin surface. The system according to various embodiments mobilizes Meibum lipids within the Meibomian glands, unblocks clogged Meibomian glands and ducts, lowers viscosity of Meibum, and improves flow of Meibum lipid to the surface of the eye and onto the tear film.

Embodiments provide a moldable heating device having a flexible and moldable three-dimensional shape which allows desired, uniform heat transfer to the area stated while the composition of the heater controls the temperature and desired length of time heat is generated and delivered to the eyelid. The reusable miniature resonance frequency stimulation vibration generator is set to a specific patient frequency stimulation to encourage the production and flow of natural tear lipid component from excitation of the Meibomian glands in the eyelids. In addition to warming the eyelid surface and Meibomian glands directly, the heat to the surrounding periorbital area increases vascular perfusion and thereby naturally increases tissue temperature. Thusly heated, the viscosity of the lipid in the Meibomian glands is decreased.

According to at least one embodiment, the addition of harmonic resonance frequency stimulation generation and applied shear forces tuned to the patient's harmonic resonance of their eyelids and Meibomian glands encourages flow of the lipids expressed in these glands, delivering this critical tear film lipid layer onto the ocular surface.

According to another embodiment, there is provided an externally powered heater in the mask along with the externally powered harmonic resonance frequency stimulation generator. The external energy source allows for circuitry in an external power module to act as a sweep range device, targeting the specific optimal point or sweep frequency stimulation to be tuned to the individual patient's eyelids during initial testing by the doctor. The initial testing device would be supplied to the doctor and would be a device with sensing elements to identify the optimally tuned harmonic resonance frequency stimulation. Once the frequency stimulation is defined and treatment provided in the doctor's office, the patient would optionally receive a reusable mask with custom harmonic resonance frequency stimulation vibration generators set to match doctor's test optimized treatment parameters. This configuration would be amenable to both single use and reusable heating warming wafer disc approaches.

According to at least one embodiment, there is provided a moldable warming device with a miniature harmonic resonance frequency stimulation vibration generator. The moldable warming device includes a moldable heating disc; the miniature harmonic resonance frequency stimulation vibration generator; a moldable coupling device; a reusable mask configured to hold the moldable heating disc, the miniature harmonic resonance frequency stimulation generator, and the moldable coupling device for use in parallel utility; and a sensor array configured to determine tuning parameters of a vibration and heating profile of a user's individual patient eyelid and periorbital three-dimensional anatomy and surface topography.

According to at least one embodiment, the moldable heating disc includes a polymer, resulting in a pliable and strong moldable material having high shape retention characteristics.

According to at least one embodiment, the moldable heating disc includes a single-use heating disc, wherein the polymer is manufactured to provide the moldable heating disc with a 20-80% pore volume, resulting in high porosity in the moldable heating disc.

According to at least one embodiment, the moldable heating disc is infused with FeOₓ heating material.

According to at least one embodiment, the FeOₓ heating material has a reaction 4Fe(s)+3O₂(g)→2Fe₂O₃(s) and includes a ratio of ingredients and produces a calibrated heater temperature at the eyelid surface for the time duration of about 5-15 minutes necessary for optimal patient therapeutic effect.

According to at least one embodiment, the moldable heating disc includes a defined pocket indentation for locating the miniature harmonic resonance frequency stimulation vibration generator.

According to at least one embodiment, the miniature harmonic resonance frequency stimulation vibration generator is preset to a defined harmonic resonance frequency stimulation for optimal patient therapeutic effect.

According to at least one embodiment, the preset harmonic resonance frequency stimulation includes frequencies of about 2 Hz-270 Hz, about 15 Hz-40 Hz, or about 30 Hz-60 Hz.

According to at least one embodiment, the preset harmonic resonance frequency stimulation for each patient is determined by a health care provider.

According to at least one embodiment, the preset harmonic resonance frequency stimulation is determined using an in-office, variable harmonic resonance frequency stimulation vibration generator configured to test available resonance frequencies to identify the individual patient's optimal therapeutic frequency stimulation for tear film stabilization and flow of Meibum from the patient's Meibomian glands.

According to at least one embodiment, the moldable warming device further includes a miniature, ultrasensitive biochemical sensor configured to detect an optimal tissue response to the preset harmonic resonance frequency stimulation.

According to at least one embodiment, the moldable warming device further includes a miniature, ultrasensitive biochemical sensor configured to map a biochemical makeup of the user's Meibomian lipids or glands.

According to at least one embodiment, the coupling device includes one of an eye patch or a reusable mask.

According to at least one embodiment, the coupling device includes one of breathable cotton, linen, bamboo, or hemp.

According to at least one embodiment, the coupling device is configured to hold the miniature resonance frequency stimulation vibration generator.

According to at least one embodiment, the coupling device includes one or two pockets for holding the moldable heating disc without the miniature resonance frequency stimulation vibration generator.

According to at least one embodiment, the coupling device further includes a vibration transfer facilitating material, such as metal wire woven into one of the breathable cotton, linen, bamboo, or hemp.

According to at least one embodiment, the coupling device further includes an electrical lead with a connector configured to drive both the miniature resonance frequency stimulation vibration generator and the moldable heating disc.

According to at least one embodiment, the moldable heating disc includes nanoparticles of ferrous or non-ferrous metals to facilitate both heat transfer and harmonic resonance frequency stimulation transfer to the user's Meibomian glands.

According to at least one embodiment, the moldable heating disc includes nanoparticles of ferrous or non-ferrous metals and nano-ceramic particles to facilitate heat transfer, resonance frequency stimulation vibration transfer, and control a heat transfer rate to the user's Meibomian glands.

According to at least one embodiment, the coupling device includes a coupling agent material including hydrogel used for transferring heat and harmonic stimulation vibration energy or movement to the eyelid surface and the user's Meibomian glands.

According to at least one embodiment, the coupling agent material transmits the mechanical response from the eyelid surface and the user's Meibomian glands to a sensor.

According to at least one embodiment, the coupling agent material is a single use, disposable sterile or non-sterile component.

According to at least one embodiment, the coupling device further includes a solution sensor integrated into the coupling device, wherein the solution sensor is configured to record a response.

According to at least one embodiment, the coupling device further includes an integrated piezoelectric device configured to record the response and compare the response to a patient subjective impression of predetermined frequency stimulation.

Embodiments provide a moldable warming device. The moldable warming device includes a heating disc, a harmonic resonance frequency stimulation vibration generator (RFSVG), a coupling device, a mask, and a sensor array. The mask is configured to hold the heating disc, the harmonic RFSVG, and the coupling device for use in parallel utility. The sensor array is configured to determine tuning parameters of a vibration and heating profile of a user's individual eyelid and periorbital three-dimensional anatomy and surface topography. The moldable warming device is configured to provide an entire eyelid surface and periorbital structures with therapeutic warmth and tuned harmonic resonance frequency stimulation vibration to mobilize Meibum lipids and stimulate flow of the mobilized Meibum lipids from the user's Meibomian glands.

According to at least one embodiment, the mask includes a shape memory bridge allowing the user flexibility forming the mask to the user's individual eyelid and periorbital three-dimensional anatomy and surface topography.

According to at least one embodiment, the coupling device is configured to contact the user's eyelid skin for heat and vibration transfer.

According to at least one embodiment, the mask includes a heat reflective material configured to reflect and direct heat from the heating disc toward the user's eyelid surface.

According to at least one embodiment, the sensor array includes a thermocouple configured to record temperature of the user's eyelid surface. In some embodiments, the sensor array includes a flexible printed circuit board where the thermocouple is embedded therein. In some embodiments, the flexible printed circuit board includes a polyimide film.

According to at least one embodiment, the sensor array includes temperature sensors, pressure sensors, moisture sensors, pH sensors, and combinations thereof.

According to at least one embodiment, the moldable warming device further includes a vibration modulation controller. The vibration modulation controller controls the stimulation of the harmonic RFSVG.

According to at least one embodiment, the moldable warming device further includes a microprocessor and a wireless transmitter. The microprocessor converts analog data generated from the sensor array into a digital data stream. The wireless transmitter transmits the digital data stream wirelessly via an antenna. In some embodiments, the wirelessly transmitted digital data stream is configured to be received by a smart device. In some embodiments, the wirelessly received digital data stream is configured to be processed by a smart device application.

Embodiments provide a method for ophthalmic eyelid therapy. The method includes the steps of applying a moldable warming device to a user's individual eyelid and periorbital three-dimensional anatomy and surface topography, generating thermal energy and harmonic resonance frequency stimulation vibration, transferring the thermal energy and the harmonic resonance frequency stimulation vibration to either the inner surface of the eyelid or an entire eyelid external skin surface and periorbital structures, and mobilizing Meibum lipids and stimulating flow of the mobilized Meibum lipids from the user's Meibomian glands. The moldable warming device includes a heating disc, a harmonic RFSVG, a coupling device, a mask, and a sensor array. The mask is configured to hold the heating disc, the harmonic RFSVG, and the coupling device for use in parallel utility.

According to at least one embodiment, the transferring step includes transferring the thermal energy and the harmonic resonance frequency stimulation vibration to an inner surface of the eyelid.

According to at least one embodiment, the mask includes a shape memory bridge allowing the user flexibility forming the mask to the user's individual eyelid and periorbital three-dimensional anatomy and surface topography.

According to at least one embodiment, the coupling device is configured to contact the user's eyelid skin for heat and vibration transfer.

According to at least one embodiment, the mask includes a heat reflective material configured to reflect and direct heat from the heating disc toward the user's eyelid surface.

According to at least one embodiment, the sensor array includes a thermocouple configured to record temperature of the user's eyelid surface. In some embodiments, the sensor array includes a flexible printed circuit board where the thermocouple is embedded therein.

According to at least one embodiment, the sensor array includes temperature sensors, pressure sensors, moisture sensors, pH sensors, and combinations thereof.

According to at least one embodiment, the method further includes the step of generating, at the sensor array, a data stream responsive to a vibration and heating profile of the user's individual patient eyelid and periorbital three-dimensional anatomy and surface topography. In some embodiments, the method further includes the steps of converting the data stream from analog to digital, and transmitting the data stream wirelessly via an antenna. In some embodiments, the method further includes the step of receiving the wirelessly transmitted data stream using a smart device. In some embodiments, the method further includes the step of processing the wirelessly transmitted data stream using a smart device application

Embodiments provide a cloud-based ophthalmic eyelid treatment monitoring system. The monitoring system includes a moldable warming device, a monitor, and a cloud server. The moldable warming device includes a heating disc, a RFSVG, a coupling device, a mask, and a sensor array. The mask is configured to hold the moldable heating disc, the RFSVG, and the coupling device for use in parallel utility. The sensor array is configured to generate a data stream responsive to a vibration and heating profile of a user's individual patient eyelid and periorbital three dimensional anatomy and surface topography. The monitor is configured to receive the data stream from the sensor array and configured to transmit the data stream to the cloud server. The cloud server is configured to process the data stream so as to determine tuning parameters of the vibration and heating profile of the user's individual patient eyelid and periorbital three-dimensional anatomy and surface topography, and configured to transmit the tuning parameters to the monitor. The monitor is configured to receive the tuning parameters from the cloud server, configured to display information related to the tuning parameters, and configured to transmit the tuning parameters to the moldable warming device. The moldable warming device is configured to receive the tuning parameters from the monitor and configured to generate thermal and vibratory stimulation energy according to the tuning parameters.

According to at least one embodiment, the moldable warming device is configured to provide an entire eyelid surface and periorbital structures with therapeutic warmth and tuned harmonic resonance frequency stimulation vibration to mobilize Meibum lipids and stimulate flow of the mobilized Meibum lipids from the user's Meibomian glands.

According to at least one embodiment, the coupling device is configured to contact the user's eyelid skin for transferring thermal and vibratory energy.

According to at least one embodiment, the sensor array includes sensors such as temperature sensors, pressure sensors, moisture sensors, pH sensors, and combinations thereof.

According to at least one embodiment, the moldable warming device further includes a microprocessor and a transmitter. The data stream generated by the sensor array is analog. The microprocessor receives the data stream from the sensor array and converts the data stream into digital. The transmitter receives the data stream from the microprocessor and transmits the data stream wirelessly. According to at least one embodiment, the monitor receives the data stream wirelessly transmitted from the transmitter.

According to at least one embodiment, the monitor is configured to wirelessly transmit the data stream to the cloud server.

According to at least one embodiment, the cloud server is configured to wirelessly transmit the tuning parameters to the monitor.

According to at least one embodiment, the monitor is configured to wirelessly transmit the tuning parameters to the moldable warming device.

According to at least one embodiment, the monitoring system further includes a cloud computing unit. The cloud computing unit utilizes artificial intelligence, big-data pattern recognition, and combinations thereof, to optimize the tuning parameters.

According to at least one embodiment, the cloud server includes a database of information including treatment data, sensor data, meta-data, patient-provided data, and combinations thereof.

Embodiments provide a cloud-based ophthalmic eyelid treatment monitoring system. The monitoring system includes a cloud server, an in-office system, and an at-home system. The cloud server is configured to determine a first set of tuning parameters related to a heating profile of a user's individual eyelid and periorbital three-dimensional anatomy and surface topography. The cloud server is configured to transmit the first set of tuning parameters to an in-office monitor of the in-office system and an at-home monitor of the at-home system. The in-office system includes an in-office mask and the in-office monitor. The in-office mask includes a first heat source. The in-office monitor is configured to transmit the first set of tuning parameters to the in-office mask for the first heat source to generate thermal energy according to the first set of tuning parameters. The at home system includes an at-home mask and the at-home monitor. The at-home mask includes a second heat source. The at-home monitor is configured to transmit the first set of tuning parameters to the at-home mask for the second heat source to generate thermal energy according to the first set of tuning parameters.

According to at least one embodiment, the cloud server utilizes artificial intelligence, big-data pattern recognition, and combinations thereof, to optimize the first set of tuning parameters.

According to at least one embodiment, the cloud server includes a database of information such as treatment data, sensor data, meta-data, patient-provided data, and combinations thereof.

According to at least one embodiment, the in-office mask further includes at least one sensor. The at least one sensor is configured to generate a data stream responsive to the heating profile of the user's individual eyelid and periorbital three-dimensional anatomy and surface topography. The in-office monitor is configured to receive the data stream from the at least one sensor and to transmit the data stream to the cloud server.

According to at least one embodiment, the at-home mask further includes at least one sensor. The at least one sensor is configured to generate a data stream responsive to the heating profile of the user's individual eyelid and periorbital three-dimensional anatomy and surface topography. The at-home monitor is configured to receive the data stream from the at least one sensor and to transmit the data stream to the cloud server.

According to at least one embodiment, the cloud server is configured to determine a second set of tuning parameters related to a vibration profile of the user's individual eyelid and periorbital three-dimensional anatomy and surface topography. The cloud server is configured to transmit the second set of tuning parameters to the in-office monitor of the in-office system and the at-home monitor of the at-home system. According to at least one embodiment, the cloud server utilizes artificial intelligence, big-data pattern recognition, and combinations thereof, to optimize the second set of tuning parameters. According to at least one embodiment, the in-office mask further includes a RFSVG. The in-office monitor is configured to transmit the second set of tuning parameters to the in-office mask for the RFSVG to generate vibrational energy according to the second set of tuning parameters. According to at least one embodiment, the at-home mask further includes a RFSVG. The at-home monitor is configured to transmit the second set of tuning parameters to the at-home mask for the RFSVG to generate vibrational energy according to the second set of tuning parameters. According to at least one embodiment, the in-office mask further includes at least one sensor. The at least one sensor is configured to generate a data stream responsive to the vibration profile of the user's individual eyelid and periorbital three-dimensional anatomy and surface topography. The in-office monitor is configured to receive the data stream from the at least one sensor and to transmit the data stream to the cloud server. According to at least one embodiment, the at-home mask further includes at least one sensor. The at least one sensor is configured to generate a data stream responsive to the vibration profile of the user's individual eyelid and periorbital three-dimensional anatomy and surface topography. The at-home monitor is configured to receive the data stream from the at least one sensor and to transmit the data stream to the cloud server.

Embodiments provide a cloud-based method for monitoring ophthalmic eyelid treatment. The method includes the steps of: applying a moldable warming device to a user's individual patient eyelid and periorbital three dimensional anatomy; generating, at the sensor array, a data stream responsive to a vibration and heating profile of the user's individual eyelid and periorbital three dimensional anatomy and surface topography; communicating the data stream from the sensor array to a monitor; transmitting the data stream from the monitor to a cloud server; processing, at the cloud server, the data stream so as to determine tuning parameters of the vibration and heating profile of the user's individual patient eyelid and periorbital three-dimensional anatomy; transmitting the tuning parameters from the cloud server to the monitor; displaying, on the monitor, information related to the tuning parameters; communicating the tuning parameters from the monitor to the moldable warming device; and generating, at the moldable warming device, thermal and vibratory energy according to the tuning parameters. The moldable warming device includes a heating disc, a RFSVG, a coupling device, a mask, and a sensor array. The mask is configured to hold the heating disc, the RFSVG, and the coupling device for use in parallel utility.

According to at least one embodiment, the moldable warming device is configured to provide an entire eyelid surface and periorbital structures with therapeutic warmth and tuned harmonic resonance frequency stimulation vibration to mobilize Meibum lipids and stimulate flow of the mobilized Meibum lipids from the user's Meibomian glands.

According to at least one embodiment, the method further includes the step of converting the data stream from analog to digital.

According to at least one embodiment, the data stream is communicated wirelessly in the communicating the data stream step.

According to at least one embodiment, the data stream is transmitted wirelessly in the transmitting the data stream step.

According to at least one embodiment, the tuning parameters are transmitted wirelessly in the transmitting the tuning parameters step.

According to at least one embodiment, the tuning parameters are communicated wirelessly in the communicating the tuning parameters step.

Embodiments provide a cloud-based method for monitoring ophthalmic eyelid treatment. The method includes the steps of: determining, at a cloud server, a first set of tuning parameters related to a heating profile of a user's individual eyelid and periorbital three-dimensional anatomy and surface topography; transmitting the first set of tuning parameters from the cloud server to at least one monitor; communicating the first set of tuning parameters from the at least one monitor to at least one mask; and generating, at the at least one mask, thermal energy according to the first set of tuning parameters. The at least one mask includes a heat source.

According to at least one embodiment, the method further includes the step of displaying, on the at least one monitor, information related to the first set of tuning parameters.

According to at least one embodiment, the method further includes the steps of: generating a data stream, at at least one sensor, responsive to the heating profile of a user's individual eyelid and periorbital three-dimensional anatomy and surface topography; communicating the data stream from the at least one sensor to the at least one monitor; and transmitting the data stream from the at least one monitor to the cloud server.

According to at least one embodiment, the method further includes the steps of: determining, at the cloud server, a second set of tuning parameters related to a vibration profile of the user's individual eyelid and periorbital three-dimensional anatomy and surface topography; and transmitting the second set of tuning parameters from the cloud server to the at least one monitor. According to at least one embodiment, the method further includes the step of displaying, on the at least one monitor, information related to the second set of tuning parameters. According to at least one embodiment, the method further includes the steps of: communicating the second set of tuning parameters from the at least one monitor to the at least one mask; and generating, at the at least one mask, vibrational energy according to the second set of tuning parameters. The at least one mask includes a RFSVG. According to at least one embodiment, the method further includes the steps of: generating a data stream, at at least one sensor, responsive to the vibration profile of the user's individual eyelid and periorbital three-dimensional anatomy and surface topography; communicating the data stream from the at least one sensor to the at least one monitor; and transmitting the data stream from the at least one monitor to the cloud server.

### BRIEF DESCRIPTION OF THE DRAWINGS

So that the manner in which the recited features, aspects and advantages of the disclosure, as well as others that will become apparent, are attained and can be understood in detail, a more particular description of certain embodiments briefly summarized above can be had by reference to the embodiments that are illustrated in the drawings that form a part of this specification. It is to be noted, however, that the appended drawings illustrate only exemplary embodiments and are, therefore, not to be considered limiting of the disclosure's scope, for the disclosure can admit to other equally effective embodiments. Like numbers refer to like elements throughout, and the prime notation, if used, indicates similar elements in alternative embodiments or positions.

FIG. 1A is a front view of a heating disc device; FIG. 1B is a side or section view of the heating disc.

FIGS. 2A-2F are graphs of the force velocity and differential pressure for various porous polyolefin compositions.

FIG. 3A is a front view of a packaged heating disc; FIG. 3B is a section view of a packaged heating disc.

FIG. 4A and FIG. 4B are a side section view and front section view heater disk showing embedded metallic nanoparticles.

FIG. 5A and FIG. 5B are a side section view and front section view heater disk showing embedded metallic and ceramic nanoparticles.

FIG. 6A is a front view of the heating disc with attached resonance frequency stimulation generator; FIG. 6B is a side view of the heating disc with attached resonance frequency stimulation generator and USB connector.

FIG. 7A is a reusable mask incorporating both a miniature resonance frequency stimulation vibration generator and permanent imbedded heater element; FIG. 7B shows an example vibration modulation controller; FIG. 7C shows an example mechanical resonator; FIG. 7D shows an example control board.

FIG. 8 is a representative far infrared front-end spot heater.

FIG. 9A is a section view of a microfluidic sensor; FIG. 9B is a front view of a microfluidic sensor.

FIG. 10A is a schematic for a dual channel solution sensing electrode; FIG. 10B is a schematic for a single channel solution sensing electrode.

FIG. 11 is a photograph of the mask prototype with the proposed 3D shape memory element.

FIG. 12A is a front view of the EyeGiene® heating device demonstrating temperature and frequency stimulation generator possible mask locations, along with in-place signal conditioner and amplifying circuitry. FIG. 12B is a top view of the original moldable heater with the new addition of a miniature resonance frequency stimulation generator located in the new pocket which is formed when the moldable heater is manufactured. The frequency stimulation generator is a reusable electrical device, which snaps into place for the duration of the one-time use or reusable heater element and is then removed before the one-time use or reusable heater element is removed. This view shows the heat shield material incorporated into the design for thermal manipulation of heater element, energy output.

FIG. 13 further demonstrates the frequency stimulation controller with a temperature controller incorporated into the unit.

FIG. 14 is an electrical schematic demonstrating the circuitry to localize and transmit sensor information via antenna to a smartphone or smart device receiver, processor, and display.

FIG. 15 is a block diagram of the path to transmit data to a smartphone or smart device receiver, processor, and display.

FIG. 16 further shows a block diagram showing a smartphone or smart device as the receiver, processor, and display.

FIG. 17A is a front view drawing of a single location temperature sensor and frequency stimulation generator. The thermocouples have wireless transmission capability and the power is provided by a localized power source, replaceable battery, or USB. FIG. 17B further shows a top view of the single point sensor showing the sensor on the eyelid side of the mask.

FIGS. 18A-18C are photographs depicting a single temperature sensor with wireless transmission capabilities.

FIG. 19 is a drawing of the front view of a multiple temperature sensor array using a flexible printed circuit for eye shape capabilities and data collection via wireless transmission to external components such as a smartphone or smart device, wireless receiver, USB, processor, or display.

FIGS. 20A and 20B show photographs of a flexible printed circuit with temperature sensors or sensor array.

FIG. 21 shows a mask with multiple sensor capability. The sensor array detects temperature, pressure, moisture, pH, and frequency stimulation. This array communicates via wireless technology to a smartphone or smart device.

FIG. 22A shows a photograph of a miniaturized temperature sensing device. FIG. 22B shows a performance graph of the miniaturized temperature sensing device. FIG. 22C shows a depiction of a miniaturized moisture/humidity sensing device. FIGS. 22D-1 and 22D-2 shows photographs of a miniaturized pressure sensing device. FIG. 22E shows a depiction of a miniaturized pH sensing device. FIG. 22F shows a photograph of the miniaturized pH sensing device.

FIG. 23 is a commercial wire schematic for a smartphone or smart device to receive process, and display data from the wireless sensor array.

FIG. 24A is a graphical representation showing a front view of a human eye. FIG. 24B is a graphical representation showing an enlarged cross-sectional view of an upper eyelid of the human eye. FIG. 24C is a graphical representation showing a cross-sectional view through a Meibomian gland of an upper eyelid of a human eye where the harmonic resonance frequency stimulation vibration mobilizes and stimulates flow of the Meibum lipids within a Meibomian gland. The mobilized and stimulated flow leads to enhanced excretion of the Meibum lipids.

FIG. 25A shows an embodiment of an in-office ophthalmic eyelid treatment and monitoring system. FIG. 25B shows another embodiment of an in-office ophthalmic eyelid treatment monitoring system.

FIG. 26 shows an embodiment of an at-home ophthalmic eyelid treatment monitoring system where the moldable warming device is connected to the cloud server via a smartphone or smart device app.

FIG. 27 shows a schematic of a cloud-based ophthalmic eyelid treatment and monitoring system including both the in-office treatment and monitoring system and the at-home treatment and monitoring system.

FIG. 28 shows a schematic of a dry eye disease smart cloud database depicting examples of inputs and outputs.

### DETAILED DESCRIPTION

While the scope of the system and method will be described with several embodiments, it is understood that one of ordinary skill in the relevant art will appreciate that many examples, variations and alterations to the systems and methods described here are within the scope and spirit of the embodiments.

Accordingly, the embodiments described are set forth without any loss of generality, and without imposing limitations, on the embodiments. Those of skill in the art understand that the scope includes all possible combinations and uses of particular features described in the specification.

As used throughout, one of ordinary skill in the relevant art would understand that the following terms can all be used interchangeably: dry eyes, dry eye disease, dry eye syndrome, evaporative dry eye, lipid deficiency dry eyes, blepharitis, Meibomian gland disease, Meibomian gland dysfunction, and MGD.

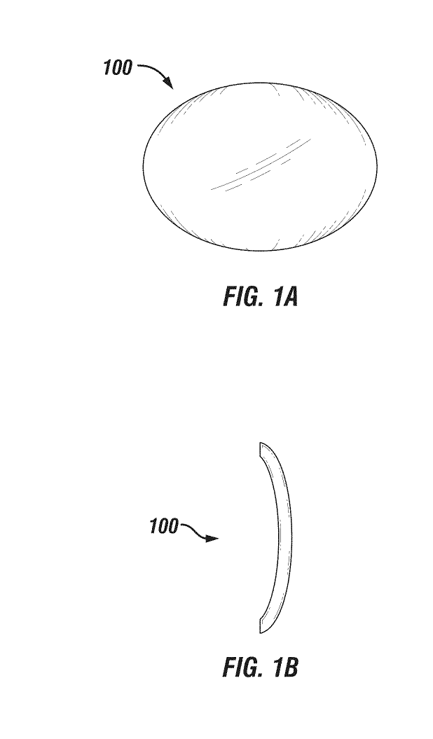

Referring now to the disclosed devices in more detail, in FIG. 1A and FIG. 1B, there is shown a heating disc 100 manufactured to fit comfortably in a reusable mask conforming to the natural shape of the closed eyelid surface, and surrounding periorbital area. A suitable shape is an oval configuration better describing the shape of the ocular surface, but can also be manufactured in rectangle or square shapes that are bigger than this minimum oval requirement.

In further detail, still referring to the devices disclosed in FIG. 1A and FIG. 1B, the heating disc 100 shows a side view of an initial curvature provided to conform the device to the closed eyelid surface by the patient when in use. The device is manufacturable such that it has a first configuration, such as an initial curvature as shown, and is conformable by the patient, physician or technician into a second configuration when in use. The material is pliable and can be easily shaped to maximize the contact area between the mask and the closed eyelid surface, to accurately match an individual user's particular orbital anatomy. For example, there will be a difference in shape for a user with a deeply set eye and deep orbit versus a user with an anteriorly placed globe. The heating disc 100 can be shaped by hand to fit a particular orbital anatomy. This approach will accurately match an individual user's particular orbital anatomy with the heating disc's high shape retention characteristics. Alternatively, light compression from the reusable eye mask will conform the heating disc 100 to the surface of the closed eyelids.

The desired heating disc integrity and heating duration are achieved by controlling disc thickness, formulation of the heating material, and porosity that allows controlled air flow to the heating material. Ideal time for application of heat is in a range from about 5 to 30 minutes, preferably from about 5 to 15 minutes and temperature at the surface of the eyelid should be between about 40 and 46° C. Example polyethylene (PE) based materials with a usable 25-60 μm pore size (PE25 through PE60) are shown in Table 1.

A nominal pore volume of 50% will allow the heating disc 100 to be reshaped or molded by the patient. The porous and moldable PE based material can have nominal pore sizes of 7-150 μm and are manufactured up to 300 μm in pore size. Another polyolefin material, polypropylene (PP), (PP-100 and PP150), shown in Table 1, is a heating disc material with 100-150 μm pore size with a smaller 45% pore volume, and can be infused with larger heater material particles for a longer disc heating time of 20-25 minutes.

TABLE 1
Porous plastic and moldable materials

| | Material Parameter | | | | | | | | | | |
|---|---|---|---|---|---|---|---|---|---|---|---|
| | PE | PE | PE | PE | PE | PE- | PP | PP | PP | PVDF | PTFE |
| | 10 | 25 | 60 | 100 | 125 | HV | 50 | 100 | 150 | 30 | 30 |
| Polymer Type | PE | PE | PE | PE | PE | PE | PP | PP | PP | PVDF | PTFE |
| Material Options | H2O/ | H2O/ | H2O/ | H2O/ | H2O/ | H2O/ | H2O/ | H2O/ | H2O/ | | |
| | SS | SS | SS | SS | SS | SS | SS | SS | SS | | |
| Nominal Pore Size | 10 | 25 | 60 | 100 | 150 | 30 | 50 | 100 | 150 | 30 | 30 |
| (μ) | | | | | | | | | | | |
| Nominal Pore | 50 | 50 | 50 | 50 | 50 | 60 | 45 | 45 | 45 | 45 | 40 |
| Volume (%) | | | | | | | | | | | |
| Air Permeability | <10 | 10-50 | 30-90 | 50-100 | 70-120 | 40-80 | 40-120 | 60-150 | 70-200 | 10-50 | 5-25 |
| (ft/min @ 1.2″ H2O | | | | | | | | | | | |
| ΔP, material | | | | | | | | | | | |
| thickness of .125″) | | | | | | | | | | | |
| H2O Intrusion | 175 | 152 | 70 | 42 | 16 | 65 | 35 | 12 | 10 | 60 | 80 |
| Pressure (mBar) | | | | | | | | | | | |
| Air Filtration (ε > 98% | .5μ | 1μ | 5μ | 15μ | 30μ | 1μ | 10μ | 50μ | 75μ | 3μ | 3μ |
| @ 50 ft/min) | | | | | | | | | | | |
| Water Filtration (ε > 95% | 5μ | 10μ | 15μ | 20μ | 90μ | 10μ | 40μ | 100μ | 150μ | 15μ | |
| @ 4 ft/min) | | | | | | | | | | | |
| Relative Raw | $$$$ | $ | $ | $ | $ | $$ | $$ | $$ | $$ | $$$ | $$$$ |
| Material Price | | | | | | | | | | | |

The heating disc 100 may be made of a broad combination of the ingredients resulting in a sufficiently rigid and strong molded material that can hold its shape, yet is easily hand moldable to the closed eyelid surface for optimal therapeutic effect. The material porosity allows a heating material to reside in the pathways with access to air at between 10-90 ft/min @ 1.2″ H₂O ΔP, where the material is between 0.125″ (3.175 mm) and 0.250″ (6.35 mm) thick with enough porosity space to adhere sufficient heating material to the support surface and internal sites. Further, the various ingredients of the disc can be substituted for different materials by shape and size to control the heating rate, total thermal energy converted and delivered, and longevity of the heat conversion.

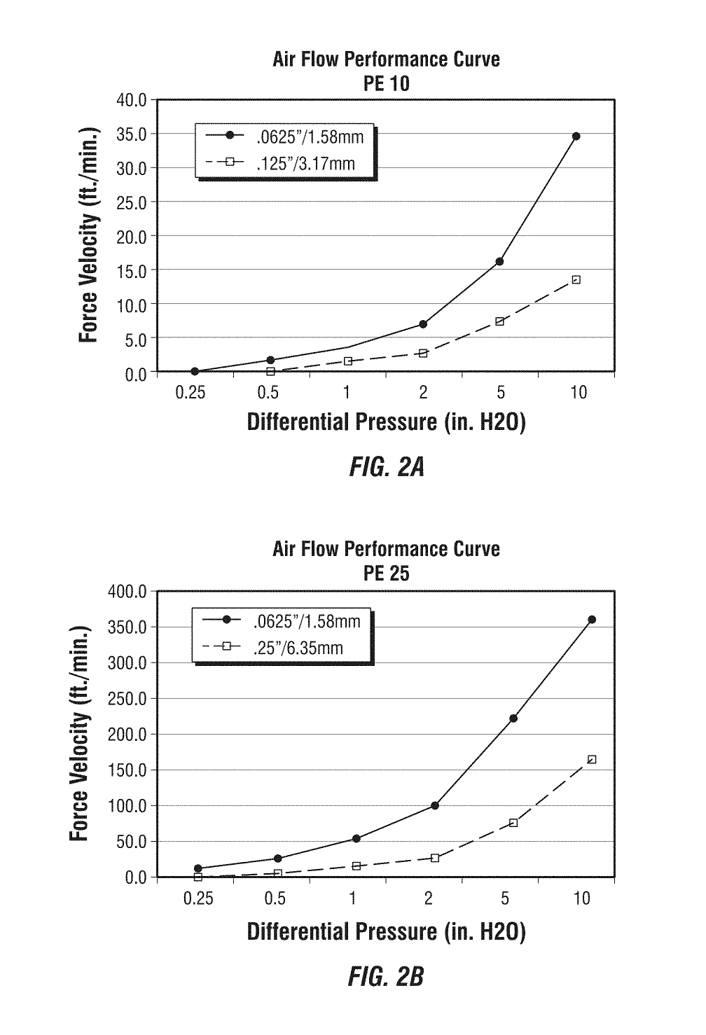

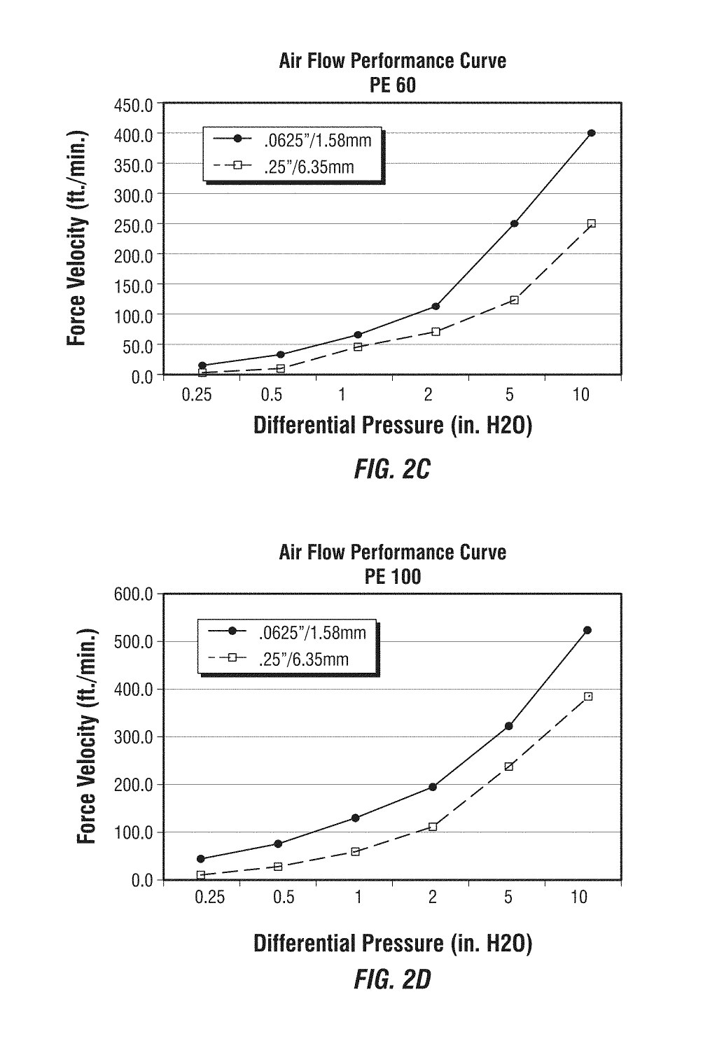

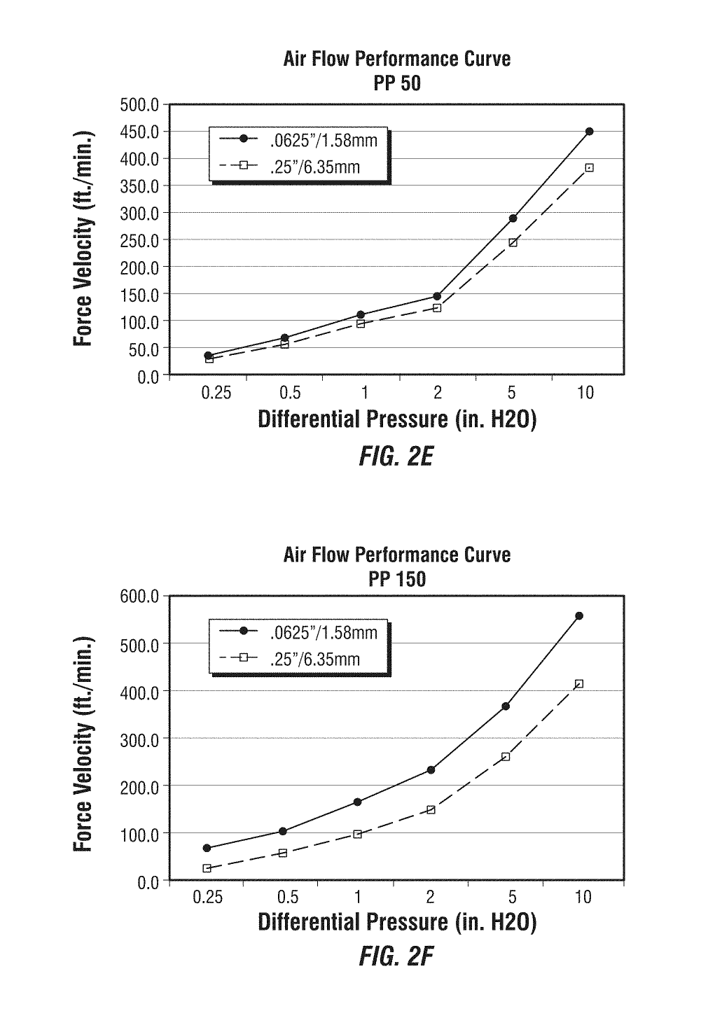

FIGS. 2A-F are graphs of the force velocity and differential pressure for various porous polyolefin compositions. Porous polyolefin compositions are used for the malleable heater disc support material described herein. FIG. 2A shows the air flow performance curve of force velocity (ft/min) and differential pressure (inches H₂O) with a thickness of 0.0625″ and 0.125″ for the PE10 material shown in Table 1. FIG. 2B shows the air flow performance curve of force velocity and differential pressure with a thickness of 0.0625″ and 0.25″ for the PE25 material shown in Table 1. FIG. 2C shows the air flow performance curve of force velocity and differential pressure with a thickness of 0.0625″ and 0.25″ for the PE60 material shown in Table 1. FIG. 2D shows the air flow performance curve of force velocity and differential pressure with a thickness of 0.0625″ and 0.25″ for the PE100 material shown in Table 1. FIG. 2E shows the air flow performance curve of force velocity and differential pressure with a thickness of 0.0625″ and 0.25″ for the PP50 material shown in Table 1. FIG. 2F shows the air flow performance curve of force velocity and differential pressure with a thickness of 0.0625″ and 0.25″ for the PP150 material shown in Table 1.

The construction details of the heating disc 100 shown in FIG. 1A and FIG. 1B are that the heating disc material be made of iron converting to ferrous oxide, with salts and inert materials resulting in the following reaction 4Fe(s)+3O₂(g)→2Fe₂O₃(s). The salts and inert materials act as reaction accelerants or retardants as needed to slow or speed the reaction and as dispersants to help create a uniform mixture within the polymer matrix. Once the reaction of 4Fe(s)+3O₂(g)→2Fe₂O₃(s) is driven to completion the individual reaction cannot easily be reversed, so each disk becomes a single use disposable product.

Still referring to FIGS. 1A & 1B, there is shown the heating disc 100 with a curvature. Although the disc appears to be solid in nature, there is porosity available to allow access of air to the surface and orifices of the heating material. The chosen porosity along with the ingredient choices and density of the disc when manufactured, and available surface area of the heating material, dictates heat conversion rates.

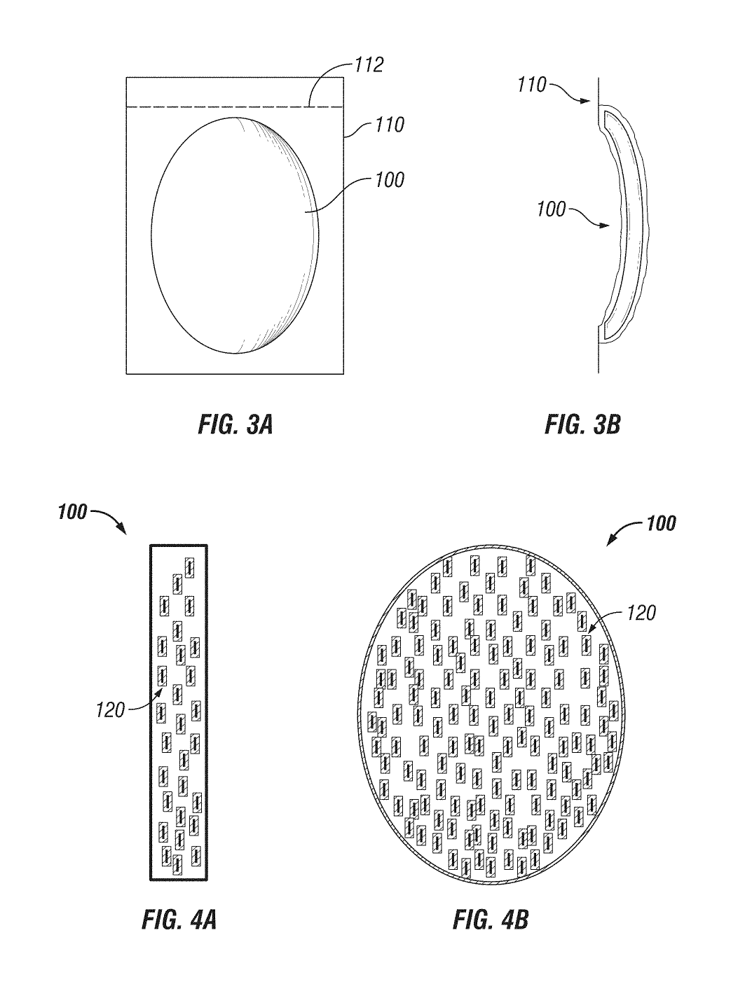

In more detail, still referring to the heating disc 100, the disc shown in FIGS. 3A & 3B includes an overwrap material or barrier layer 110 for shipping and product storage until used by the patient. The barrier layer 110 provides an extended shelf life of at least 3 years and is needed to keep H₂O and air away from the heating disc 100 to prevent premature reaction of the heating material. A pouch tear strip 112 is provided where a user may tear the barrier layer 110 for actual used of the heating disc 100. Water Vapor Transmission Rate (WVTR) measures the transmission of water vapor through a material and is determined using a modulated Infrared Sensor ASTM F1249 Test Procedure. WVTR is measured in either grams/100 in²/24 hours or grams/m²/24 hours (according to the standard ASTM—E398). Oxygen Transmission Rate (OTR) is the measurement of the amount of oxygen gas that passes through a material over a given period of time and is determined using a Coulometric Sensor ASTM D3985, ISO 15105 Test Procedure. OTR is measured in either cm³/m²/24 hours or cm³/100 in²/24 hours. The barrier layer preserves the desired properties of the disc for at least three years. Lower OTR and WVTR rates are preferred. An example of materials with good storage properties are WVTR rates of 0.3 g/100 in²/24 hours @ 37.8° F.=90% RH, for PP and 0.35 g/100 in²/24 hours @ 37.8° F.=90% RH, for high-density PE (HDPE). While very good for WVTR, these have less attractive OTR rates at 150 cm³/100 in²/24 hours @ 25° C., for PP and 110 cm³/100 in²/24 hours @ 25° C., for HDPE. Typical long-term barrier layers 110 also include a layer of aluminum foil. This is a good example of why multi-layer commercial films are used for the product overwraps which include preferred transmission rates for both WVTR and OTR characteristics. When combined, these characteristics achieve longer shelf lives for three-year protection of a product sensitive to water and oxygen.

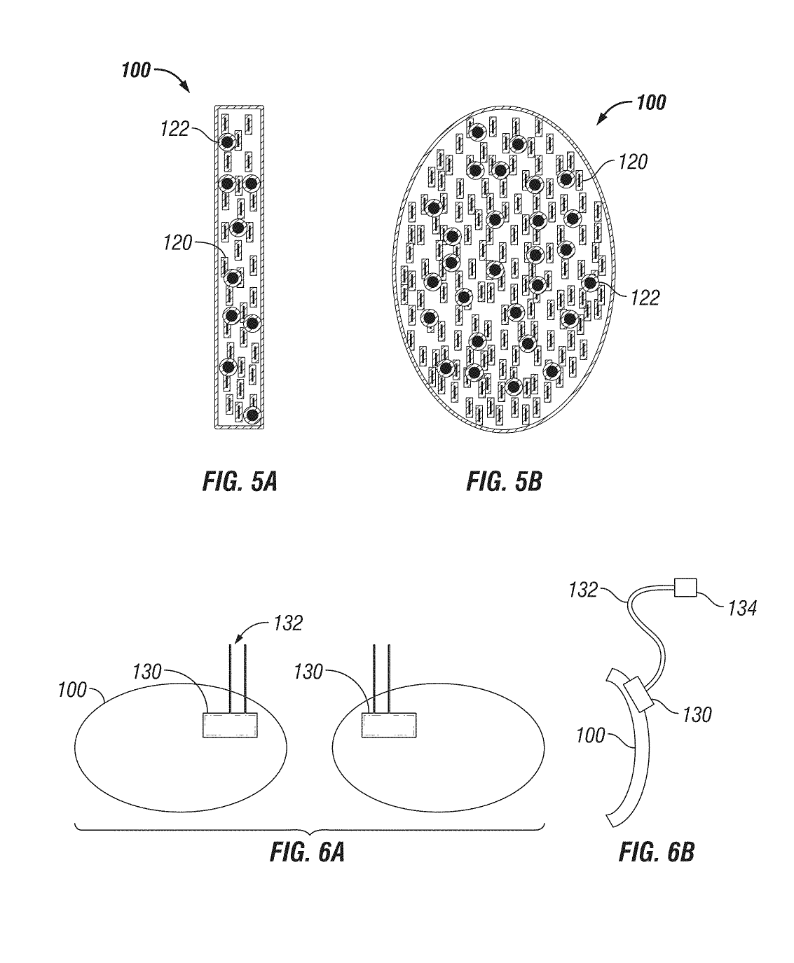

In further detail, referring to the heating disc 100 of FIGS. 4A-4B and FIGS. 5A-5B, the construction of the disc, meaning its shape, materials of construction, ingredient ratios of construction material, surface area, density and porosity make this disc a unique moldable heater product. Furthermore, the heating disk polymer support material, during manufacture, can be infused with nanoparticles of silica or metals to store and then release the heat generated by the FeOₓ heating material. This combination of components in the polymer matrix can be titrated and calibrated to transfer heat rate across the supporting materials and mask at a desired rate. Aluminum or other metal nanoparticles 120 are good heat conductors where the polymer is not. Silica or ceramic nanoparticles 122 act as a heat sink to store and slowly release the heat energy. Adding nanoparticles 120, 122 to the polymer, particularly the metals 120, also enhances desirable vibration transfer characteristics. The nanoparticles 120, 122 participate in structural integrity, also enabling a degree of structural pliability that allows for customized molding of the product to the individual patient's closed eyelid surface topography, as well as greater longevity of use for the product. This also translates into better product survival during transportation and during customer use. Polymer choices with good flexibility, shape memory, permeability and good heat transfer for desirable product characteristics can be determined from Table 1.

According to at least one embodiment, there is provided a heating disc 100 with harmonic resonance frequency stimulation vibration which is designed for use or reuse in a treating physician's office. Referring to FIGS. 6A-6B, the heating disc 100 includes resonance frequency stimulation vibration generators (RFSVG) 130 to transfer shear forces or vibrational energy to the surface of the eyes. Those RFSVGs 130 could be a single unit or one for each eye. The heating disc 100 is designed to hold the RFSVG 130 and fit comfortable over the patient's head and apply gentle pressure sufficient to transfer resonance frequency stimulation energy to the closed eyelids, either to the inner surface of the eyelid or on the entire external eyelid skin surface. To transfer the energy (heat and vibration) directly to the eyelid surface, a sanitary and disposable single use coupling device contacts the patient's skin. Energy and control is provided to the heating disc 100 via lead wires 132 and a micro USB 134 or other suitable connector from a mobile control module.

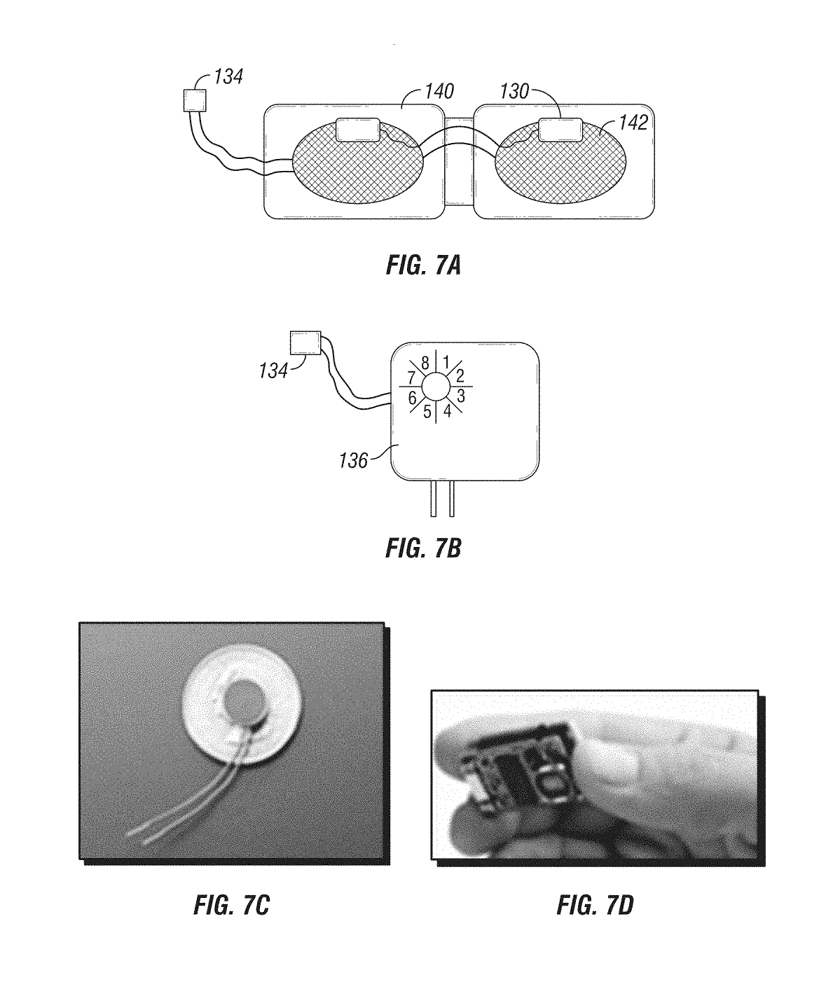

According to at least one embodiment, there is provided a heating mask 140, as shown in FIGS. 7A and 7B, with harmonic resonance frequency stimulation vibration which is designed for use or reuse in a treating physician's office. For purposes of illustration, a resusable heating mask 140 will be described in the following embodiments. Referring to FIG. 7A, the heating mask 140 contains a built-in heater element 142 or individual elements for each eye. The heater element 142 can include the heating disc 100. The system further includes RFSVGs 130 to transfer shear forces or vibrational energy to the surface of the eyelids. Those RFSVGs 130 could be a single unit or again one for each eye. The heating mask 140 is designed to hold these components and fit comfortably over the patient's head and apply gentle pressure sufficient to transfer resonance frequency stimulation energy to the closed eyelids, either to the inner surface of the eyelid or over the entire eyelid surface. To transfer the energy (heat and vibration) directly to the external eyelid skin surface, a sanitary and disposable single use coupling device contacts the patient's skin. Energy and control is provided to the heating mask 140 via a micro USB 134 or other suitable connector from a mobile control module. As shown in FIG. 7B, the heating mask 140 can include a vibration modulation controller 136 connected via USB 134.

The details of the heating mask 140 in FIG. 7A show a built-in resistance heater element 142 or metal wire woven into the mask designed with a heating element length to convert enough electrical input to the appropriate eyelid at optimum temperatures as detailed above. The resistance heater wire or metal wire can be woven into the fabric or structure of the heating mask 140 and has dual purposes: one is to generate heat when electrically energized and the other is to serve as a good vibration conductor. In some embodiments, the heater element 142 can include the heating disc 100. The resistant heater element 142 can be a single, moldable heater mesh having a shape similar to the heating mask 140.

According to at least one embodiment, power supply to the resistance heater can be accomplished for example through the USB connection 134 to a micro drive or mobile control module. Alternate power sources include disposable and rechargeable batteries. These batteries could be placed into the reusable mask if desired to eliminate cords extending from the reusable mask. A micro drive control board controlling the heater and resonator functions could be powered from a single supply voltage of 8-48 VDC, offering up to 100 W of peak power without any additional heat-sink. FIG. 7D is an example control board sold by Ingenia Motion Control. These boards combine the controller, drive, and stand-alone capability into a single unit with an incredibly small footprint. The control board preferably resides in a mobile system controller that is easy to carry around the physician's office. The mobile controller might combine the controller, drive, and stand-alone capability into a single unit with a small footprint. This is but one example of “off the shelf” components available to combine with the heating mask 140 and complete the frequency stimulation set-up and control of the heating mask 140.

According to at least one embodiment, the miniature RFSVG 130 induces a vibration through the coupling device to the surface of the eyelid. The control of vibration may include amplitude, a width, frequency, and where one or more of these parameters may be varied over the treatment period. The resonance vibration may have a frequency stimulation between about 2 Hz to about 270 Hz, between about 15 Hz to about 40 Hz, or between about 30 Hz to about 60 Hz. The resonance vibration may include a current having a pulse width or duty cycle between about 20% to about 80%. Vibration having the above-mentioned parameters may be used to treat one or more conditions, such as dry eye. Ideally in the physician's office, the controller would run through a range of pre-established frequencies and patterns. This range is to determine an individual patient's best response of resonance frequency stimulation to the applied vibration. This resonance frequency stimulation is the condition best suited to excite and mobilize an individual's flow of the Meibum lipids from the Meibomian glands. In some embodiments, anthropomorphic features and other characteristics of the patient, for example, eyelid laxity helps in the determination of the patient's personalized resonance vibration frequency. Non-limiting examples of anthropomorphic features and other patient characteristics include eyelid laxity, eyelid dimensions, eyelid mass, eyelid thickness, patient's race, patient's age, patient's sex, and any history of eyelid surgery. Non-limiting examples of anthropomorphic features and other patient characteristics also include MGD status, such as the percentage of clogged or plugged Meibomian glands, the degree of truncated Meibomian glands, and the quality of the Meibum lipid (i.e., thickness, turbidity, and clarity).

According to at least one embodiment, the tunable RFSVG 130 for the heating mask 140 may be provided by a number of different sources including sonic generators, electrodynamic or mechanical (such as cell phone vibrators) vibration generators. In some embodiments, the source is relatively quiet and able to deliver the vibrational energy through the disposable patient contacting coupling device to the underlying tissue. According to the concept of finding resonance to the patient's Meibomian glands and blocked oil glands, the frequency stimulation may be adjustable and tunable. There are a number of miniature vibration modules such as Adafruit, shown in FIG. 7C, which have the recommended range for patient individual tuning capabilities. This particular module has a 0-5 VDC range running at 11,000 rpm at the top end at 100 mA. The range may be reduced to 0, by reducing the voltage to the module, for example: 3 VDC @ 60 mA is linear and at 60% or 6,600 rpm. The power requirement is very low and may be operated by remote supplied energy or in-eye-mask supplied battery power.

According to at least one embodiment, direct heating of the eyelids and adjacent areas may be achieved by weaving a resistance NiChrome heater wire as the heater element 142 into the heating mask 140 as shown in FIG. 7A. The energy to heat the heating mask 140 to the desirable temperature may be provided by a USB connector 134. The resistance across the NiChrome heater wire dictates the heat converted by the amount of energy provided. The heating mask 140 has a compartment, or slip on top of the heater area, for the imbedded RFSVG 130. The heat control from the heater wire 142 can also be controlled by the same type device controlling the RFSVG 130.

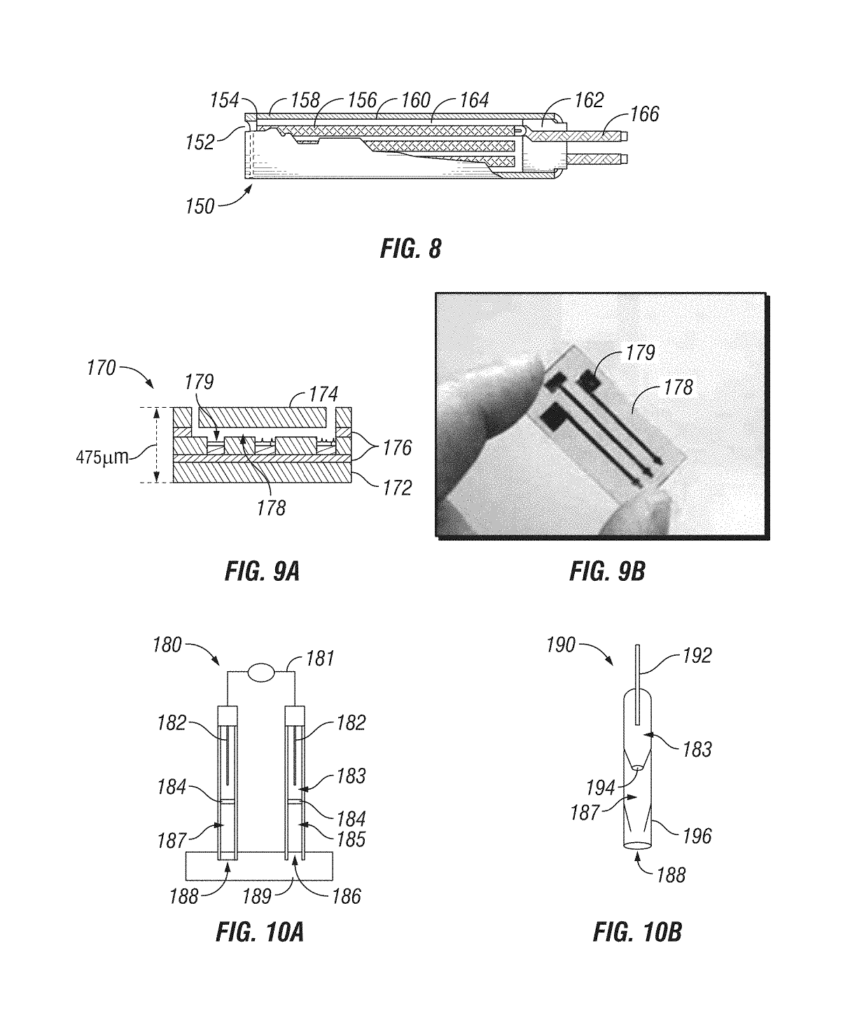

Referring to FIG. 8, the patient's Meibomian gland can also be warmed effectively and comfortably by use of a far infrared front end spot heater 150. Far infrared radiation may be directed to a precise location and the target area warmed to a precise temperature of about 40-46° C. Radiation is the most prevalent source of heat transfer in our universe and the Stefan-Boltzman law of radiation states that as the temperature of a heat source is increased, the radiant output increases to the fourth power of its temperature. Although not to be bound by any theory, this may suggest that far infrared targeted heating is a logical approach to Meibomian gland warming.

According to at least one embodiment, the far infrared front end spot heater 150 is constructed to radiate heat from the far infrared end seal 152 made of heat transmitting material (thin metal face or substitute). Heat is transferred to the far infrared end seal 152 by a conducting plug 154. This plug 154 is in contact with the end seal 152 and is a designed mass of conducting material for storing and releasing the heat converted by a heating element wire 156. The exterior or sides of the spot heater 150 include heat resistant insulation material 158 allowing a user to comfortably hold the spot heater 150 without risk of uncomfortable temperature exposure. A thermocouple (not shown) might also be employed with this device and integrated into the spot heater 150 properly. The interior of the spot heater 150 includes a conducting packing material 160 all the way to the tip or the plug 154 through a ceramic cap 162. The heating element wire 156 is supported in the spot heater 150 by ceramic element supports 164 that function in a stability capacity providing little movement and adding longevity to the spot heater device 150. The electrical leads 166 are fed through the ceramic cap 162 providing support for the electrical leads 166 and temperature barrier characteristics. The insulated electrical leads 166 includes insulated electrical wire with lead lengths ending in a USB connector 134 for operating the spot heater 150 in the physician's office.

According to at least one embodiment, the heating mask 140 includes soft, comfortable fabric like materials with an adjustable band to help the heating mask 140 reside in the appropriate location on the eyes. A moldable coupling device is a component for the heating mask 140 to provide a sanitary, possibly sterile, skin contacting surface for individual patient use. This single use, disposable coupling device will transfer the generated thermal and vibration energy generated by the heating mask 140 effectively to the eyelid surface. In some embodiments, the coupling device is composed of hydrogel, similar to a hydrogel dressing, possibly contained in a support structure or quilted construction to assure even distribution and intimate contact across the skin contacting regions. The hydrogel composition and water are controlled to best achieve this transfer, and add a controlled amount of moisture to the eyelids and lashes, with the added benefit of loosening debris on the eye lashes. According to at least one embodiment, the hydrogel layer makes direct skin contact. In alternate embodiments, the hydrogel could be constrained behind a thin moisture permeable barrier layer. In other embodiments, the coupling device is composed of a hydrogel sheet, and more particularly includes tea tree oil for treatment of, for example, demodex (i.e., mites) infestation of the eyelashes, which is common in blepharitis, Meibomian gland dysfunction, and dry eye disease.

Construction of the coupling device would allow hand molding to an individual's face, periorbita, and features or gentle reforming could be applied from pressure by the eye heating mask 140. The disposable coupling device would be easily replaceable in the heating mask 140 for use by a new patient. The coupling device would be prepared for long term storage using the barrier layer technologies described for the heating disk 100 and could be sterilized to a 10⁻³ or higher sterility assurance level (SAL).

As explained with the heating disk 100 above, this hydrogel layer could incorporate a mixture of particles to facilitate well dispersed heat transfer, heat sinking and bi-directional vibration energy transfer.

Alternatively, the coupling device could be made from thin layers of natural materials and fibers to create a comfortable and breathable surface against the skin. The heating mask 140 could be any number of fiber materials known to be breathable, such as cotton, linen, bamboo, or hemp. Other cloth fabrics from synthetic materials are also breathable and moisture transportable. Non-limiting examples include base layer clothing made from polyester and polypropylene. Filler materials inside the coupling device could be also made of breathable, natural fillers. The filler material may allow the heat to pass to the contact surface but also the vibration energy. Possible natural fillers, in small chunks or fibers, include bamboo fiber, small dried beans, quinoa, rice, and hemp. Size and size distribution of the filler material can be optimized to determine the best options for transmitting the vibration energy. Also possible are quilted fabric layers using various fillers to provide the loft in the quilt and non-woven felt materials.

According to another embodiment, the coupling device would apply moist heat to the surface. A source for the moist water vapor could be the hydrogel. As heat energy from the heating mask 140 transfers to the coupling device, water in the hydrogel or natural filler turns to vapor and crosses a moisture permeable barrier to the contact surface.

Alternately, reservoirs of water could be constructed into the coupling device to interact with the heat source.

According to at least one embodiment, a microfluidic enabled sensor shown in FIGS. 9A and 9B can be included at the patient's eye lid interface and be responsive in real time to track changes in Meibomian fluid flow. Changes in flow rate are induced by variation in vibration frequency stimulation from the RFSVG; the objective being to determine the best vibration parameters for an individual patient. The sensor provides analysis of very small samples and environments such as the Meibomian gland with the ability to measure very small change in flow. The chemical sensor is an ultra-sensitive yet simple sensor integrated into a microfluidic device, incorporating polymer-based Meibomian fluid selective liquid-contact and polymer-based solution-selective electrodes. The target component in the Meibomian fluid for the sensor analysis could be specific proteins, lipids, or other biomarkers produced with the flow of the fluid. In-situ sensors enable analysis of very small samples and environments such as the Meibomian gland at work with the ability to realize potentiometric output from very small changes of fluid flow utilizing liquid-contact electrodes, such as electrodes shown in FIGS. 10A and B. It is possible to incorporate the miniature RFSVG and the miniature integrated chemical sensor into the same device.

According to at least one embodiment, one form of the chemical sensor 170, shown in FIGS. 9A and B, has a number of layers formed by polymethylmethacrylate (PMMA) construction. This sensor has a lower PMMA layer 172, a top PMMA layer 174, a sensor liquid entrance, pressure sensitive adhesive (PSA) 176, conducting polymers, thermal pressure laminating, and CO₂ laser-generated 400 μm width fluid flow channels 178. The height of the microfluidic chemical sensor 170 is about 475 μm. A solution reactive material may be needed in the channels for detection on the Meibomian gland fluid selective catalyst layer 179. The deposition of solution reactive materials in the channeling is accomplished by electrode sputtering, if metallic. This process is well known in the art but has yet to focus on Meibomian gland issues as a target for patients until now. In this configuration, the data recorded relates to the presence of fluid.

Referring to FIG. 10A, a miniature solution selective electrode (SSE) 180 with integrated dual chemical sensors with potentiometric output is displayed. The chemical sensor uses a reference electrode 181 including an inner reference half-cell 182, a reference solution 183, a diaphragm 184, bridge solution 185 with the diaphragm 184, a capillary or sleeve 186 at the entrance. The SSE 180 includes the inner reference half-cell 182, the reference solution 183, the diaphragm 184, an inner filling solution 187, and a solution selective membrane 188. The sleeve 186 and the solution selective membrane 188 are in contact with a fluid sample 189. This miniature chemical sensor, when placed on the surface of the Meibomian gland, detects the duration of flow for patient diagnosis. Electric potential output from the SSE 180 is connected to a data acquisition module (not shown) capable to retrieve data up to 100 Hz speeds, real-time. This may allow real-time data mapping of the patient's Meibomian gland fluid, proteins, biomarkers, or lipids, which all are tracked to define each patient's individual characteristics and medical needs.

According to at least one embodiment, a single element version of the miniature integrated chemical sensor 190 with potentiometric detection, shown in FIG. 10B, is also applicable. It includes a solution sensing wire material 192, the reference solution 183, a separation plug 194, the inner filling solution 187, a SSE body 196, and the selective membrane 188. This single element miniature integrated chemical sensor 190 may be placed on the Meibomian gland and record data at the same rate as the SSE 180.

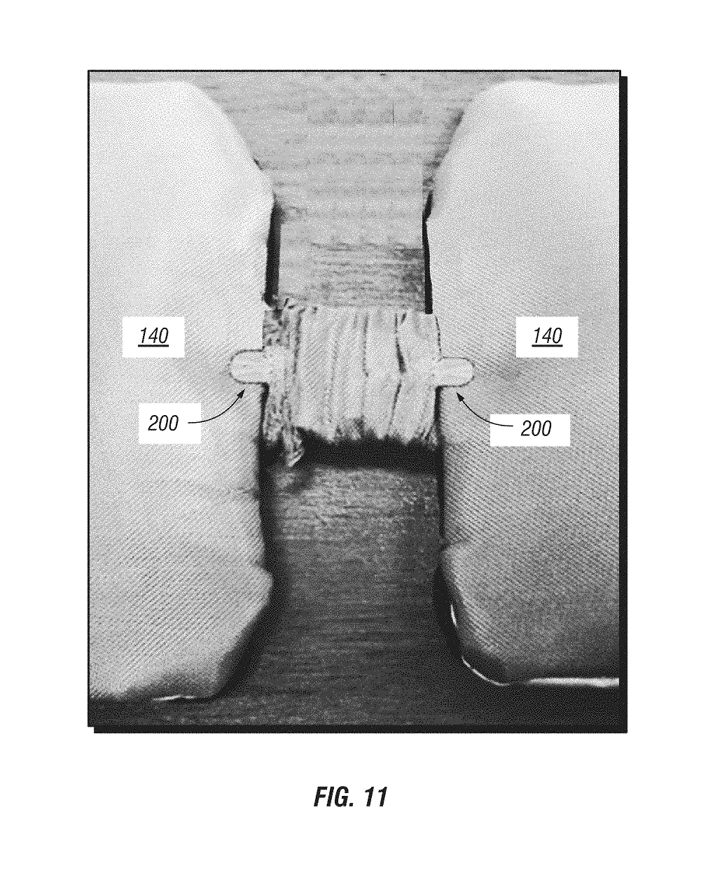

Referring now to the disclosed devices in more detail, in FIG. 11, there is shown the center connecting part of the mask, demonstrating a shape memory bridge 200 component allowing the patient flexibility forming the heating mask 140 to the patient's particular facial and periorbital features and surface topography. This allows better contact surface area by the mask's eye side surface to the eyelid thus achieving better heat transfer to the eyelid surface. According to at least one embodiment, the heating device 100 is manufactured to fit comfortably in the heating mask 140, conforming to the natural shape of the closed eyelid surface, and surrounding periorbital area. One suitable shape is an oval configuration relating to the shape of the ocular surface, other shapes, such as rectangle or square shapes that are larger than the oval shape, are also suitable.

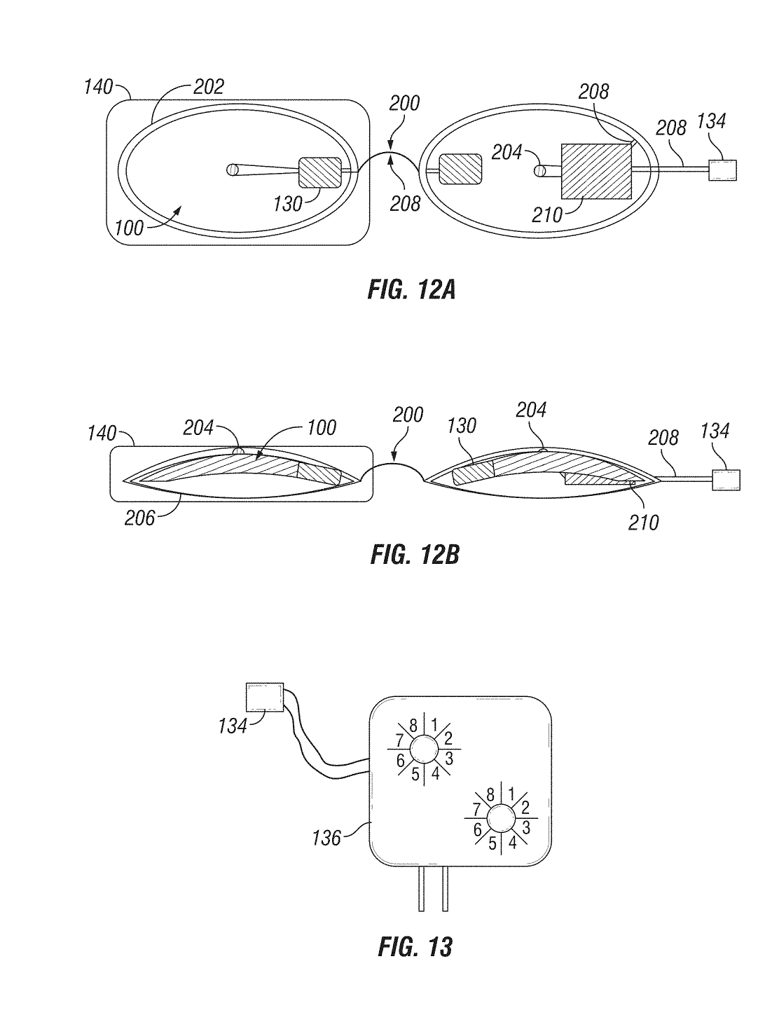

In further detail, referring to the devices disclosed in FIG. 12A and FIG. 12B, there is provided a front and top view of the moldable heating device 100 incorporated in the heating mask 140 with the RFSVG 130 located in one possible location in the moldable heater device 100. The drawing further details the layer of material against the skin 202 allowing better heat and vibration transfer via conductive heat transfer to the skin surface from the heating mask 140. Also shown is the shape memory bridge 200 allowing the patient flexibility forming the heating mask 140 to the patient's particular facial and periorbital features and surface topography. The heating mask 140 configuration shown in FIGS. 12A and 12B incorporates a single thermocouple 204 in each side of the heating mask 140 along with the RFSVG 130. In some embodiments, the mask material located on the outside layer of the heating mask 140 is made of heat reflective material 206 and is purposed to reflect and direct heat from the heating device 100 inward toward the eyelid surface. Reducing heat loss to the outside of the heating mask 140 has advantages such as better temperature control, patient comfort, and overall better temperature distribution over the eyelid surface area. The single thermocouples 204 are imbedded in the inner most layer against the eyelid and record the temperature in that location. The two dissimilar conductors produce a temperature dependent voltage carried via mask wiring 208 to the thermocouple multiplexing circuit board 210 where the signal is conditioned and carried via the USB connector 134 to a remote monitoring device or display for viewing or analysis. In further detail, referring to the devices of FIGS. 12A and 12B, the location of the RFSVG 130 is pre-positioned by the manufacturing process of the heating device 100. The pocket is sized for the RFSVG 130 and associated power sources and connections. The RFSVG 130 can be a reusable electrical device, which snaps into place for the duration of the one-time use or reusable heating device 100 and is then removed before the one-time use or reusable heating device 100 is removed.

FIG. 13 shows a rendition of further capabilities of the RFSVG 130 vibration modulation controller 136 incorporating a temperature controller for the electrical heater element 142, described in FIGS. 7A-7D. The vibration modulation controller 136 can be operated by the user or the physician. Strict control over the electrical heater element 142 allows flexibility over the range of temperatures used in dry eye therapy. The signal is carried out and power is provided via the USB connector 134.

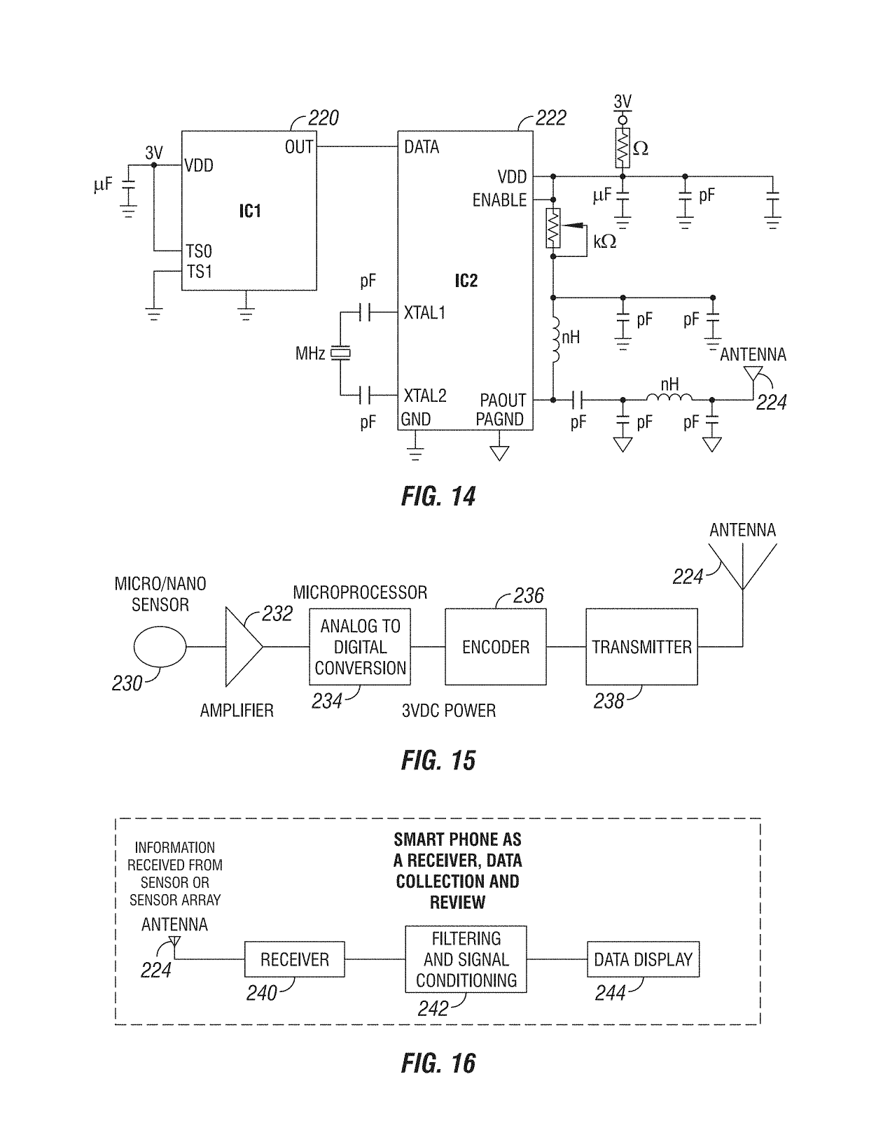

FIG. 14 shows an example electrical schematic showing the circuitry required to localize and transmit sensor information via antenna to a smartphone or smart device receiver, processor, and display. This technology has been well developed and is used to communicate collected data from sensors and sensor arrays via a smartphone or smart device app to a smartphone or smart device receiver, processor, and display. Sensor information collected by IC1 220 is transmitted to IC2 222. IC2 222 processes sensor information and further transmits the processed sensor information wirelessly via antenna 224.

FIG. 15 and FIG. 16 are block diagrams showing the path to transmitting and receiving sensor data to a smartphone or smart device receiver, processor, and display. Block diagrams are included as an assistance feature to better understand or conceptualize the embodiments disclosed herein.

According to at least one embodiment, referring to FIG. 15, analog data generated by the sensors or sensor array 230 is amplified via an amplifier 232 and converted into digital data streams via an analog-to-digital conversion (ADC) microprocessor 234. The digital data streams are encoded via an encoder 236 and then transmitted using a transmitter 238 wirelessly via an antenna 224.

According to at least one embodiment, referring to FIG. 16, wirelessly transmitted data, originated from the sensors or sensor array 230, is received using a receiver 240 via antenna 224 and is then filtered and conditioned for data display 244 using a signal filter and conditioner 242. In some embodiments, a smartphone or smart device is the receiving device. The smartphone or smart device is capable for filtering and conditioning the wirelessly transmitted data, and is also capable for displaying the processed data.

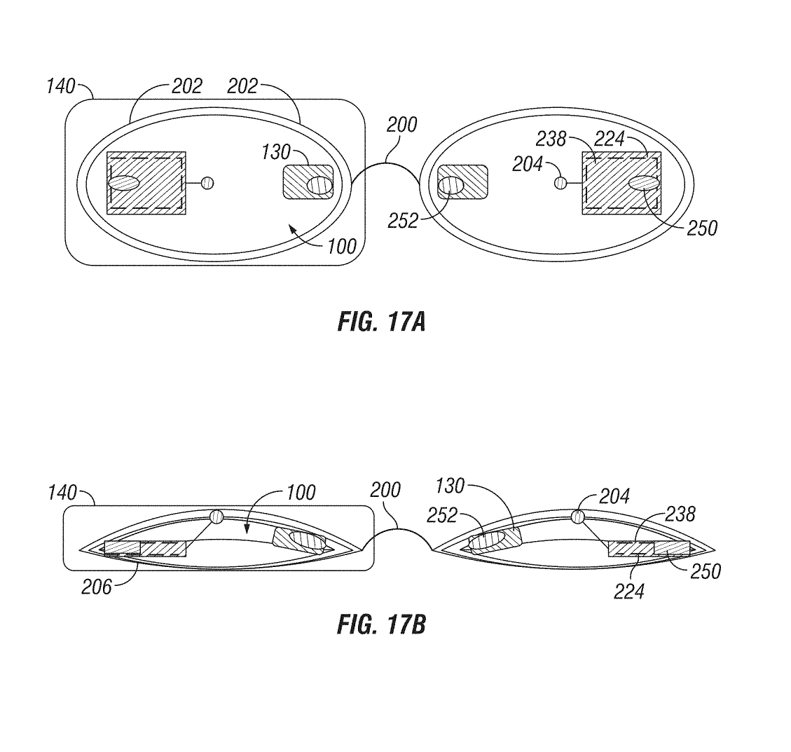

FIGS. 17A and B, still referring to the device in FIGS. 12A and B, show a single temperature sensor 204 location and RFSVG 130 in each ocular cavity along with localized transmission power source 250 and RFSVG power source 252 to energize the circuitry and frequency stimulation generator, respectively. In some embodiments, the wireless transmitter 238 is powered locally by a 3 VDC one-time-use or replaceable battery 250 where the thermocouple 204 data is transmitted via an antenna 224 to the patient's smartphone or smart device. The temperature sensor 204 is located in a forward proximity position to the eyelid for reliable and accurate temperature data collection. In some embodiments, the shape memory bridge 200 has no additional electronic connections in the wireless transmittal version. This configuration could also employ a remote power source via USB 134 where the shape memory bridge 200 would carry additional circuitry, corresponding to mask power and communication wiring 208, to transmit the temperature data.

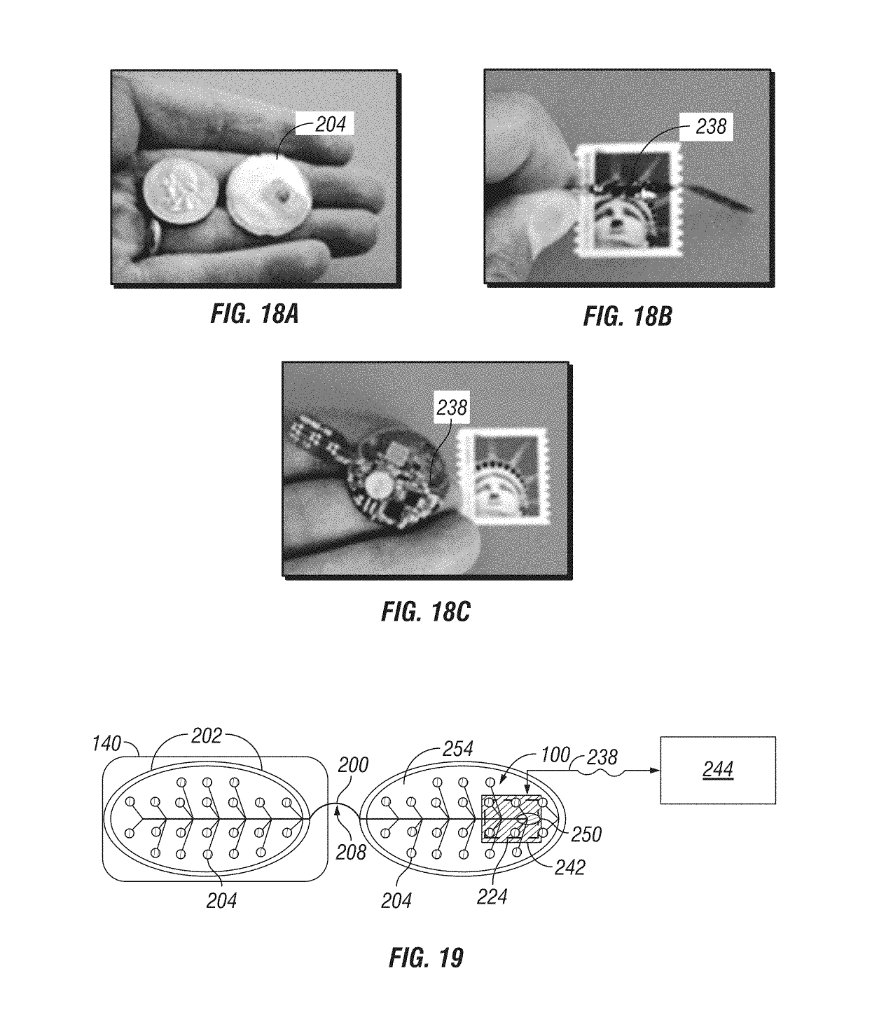

FIGS. 18A-18C show photographs of the microtechnology for collecting temperature data, for conditioning and transmitting the data to a smartphone or smart device. FIG. 18A shows the single thermocouple 204 next to a quarter for size comparison. FIG. 18B shows a side view of the data transmitter 238. FIG. 18C shows a front view of the data transmitter 238. Postage stamps are displayed for size comparison in FIGS. 18B-18C. The micro-components are easily integrated into the heating mask 140 for enhanced characteristics and utility of the proposed device.

FIG. 19, still referring to the device in FIGS. 12A-12B, shows a configuration with multiple thermocouple 204 positions, signal conditioning 242, and wireless transmission 238 of temperature data to a smartphone or smart device, wireless receiver 240, processor, and data display 244. Also provided is a flexible printed circuit board 254, which includes the thermocouples 204. In some embodiments, taking more temperature points in the given area provide a better temperature profile of the amount of energy and energy distribution produced by the heating device 100. The heating device manufacturer may focus on a more precise heater product and tailor the heater device 100 to the individual patient. In other embodiments, still referring to FIG. 19, a micro-frequency stimulation generator, such as the RFSVG 130, may be incorporated, which may be powered by a remote or localized power source, a replaceable battery, or USB connection 134.

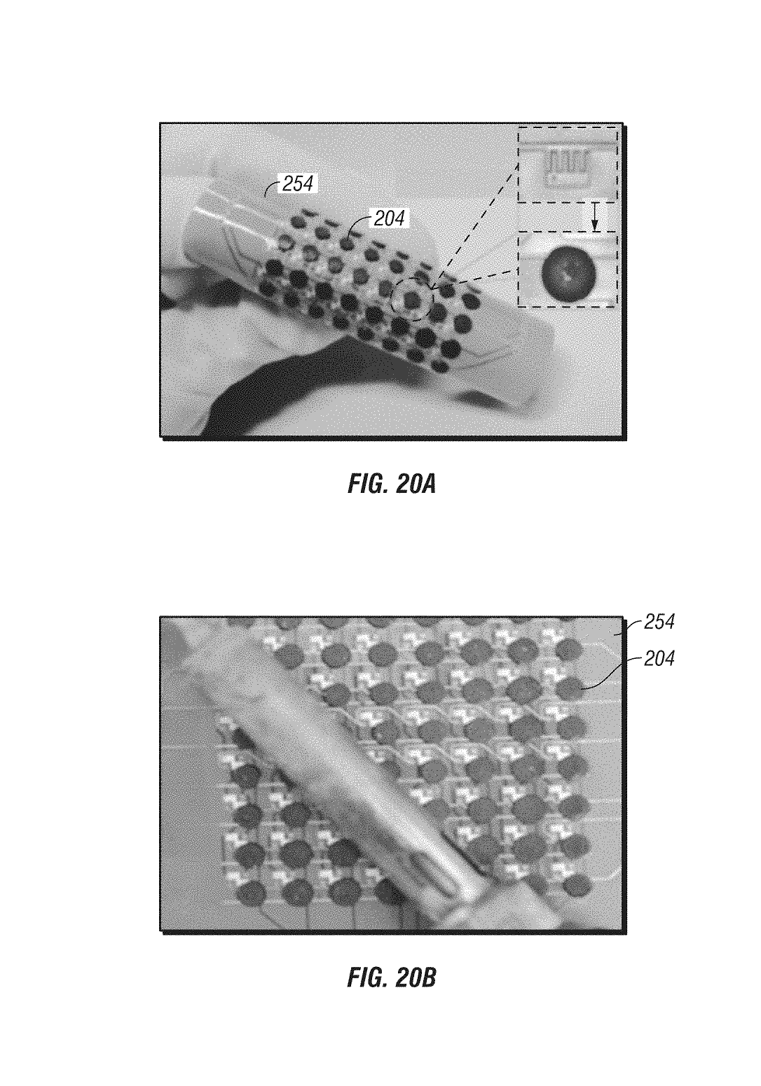

FIGS. 20A and 20B show a flexible printed circuit board 254 with closely aligned temperature sensors 204. This embodiment demonstrates how flexible the heating device 100 may be and how it may be used in the heating mask 140. In some embodiments, graphite-polydimethylsiloxane composite is dispensed on flexible polyimide films. A sensor array 204 may be, for example, an array with 64 sensing cells in a 4×4 cm² area. Interdigitated copper electrodes are patterned on the flexible polyimide substrate to determine the resistivity change of the composites subjected to ambient temperature variations. The flexible circuit operates with no signs of degradation in temperatures from about 30° C. to about 110° C.

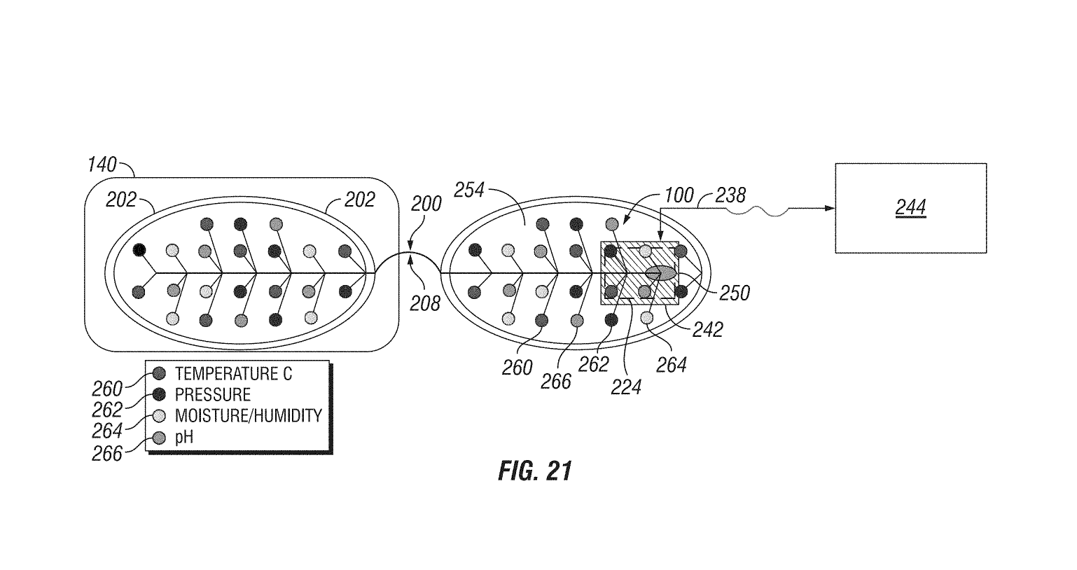

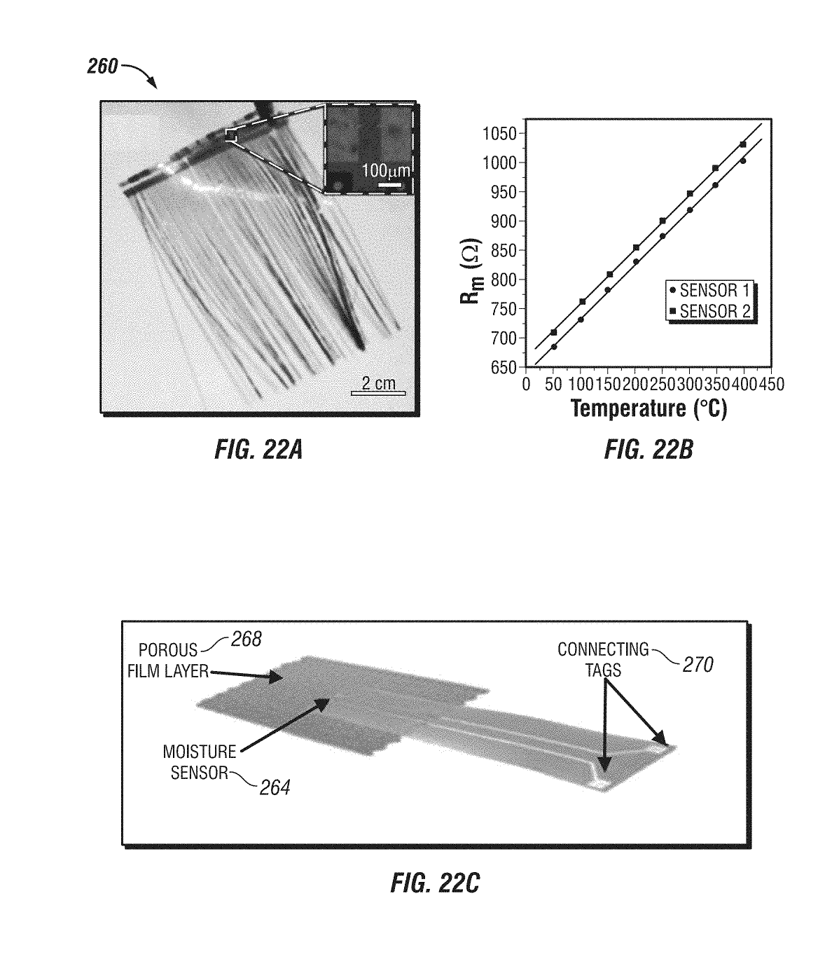

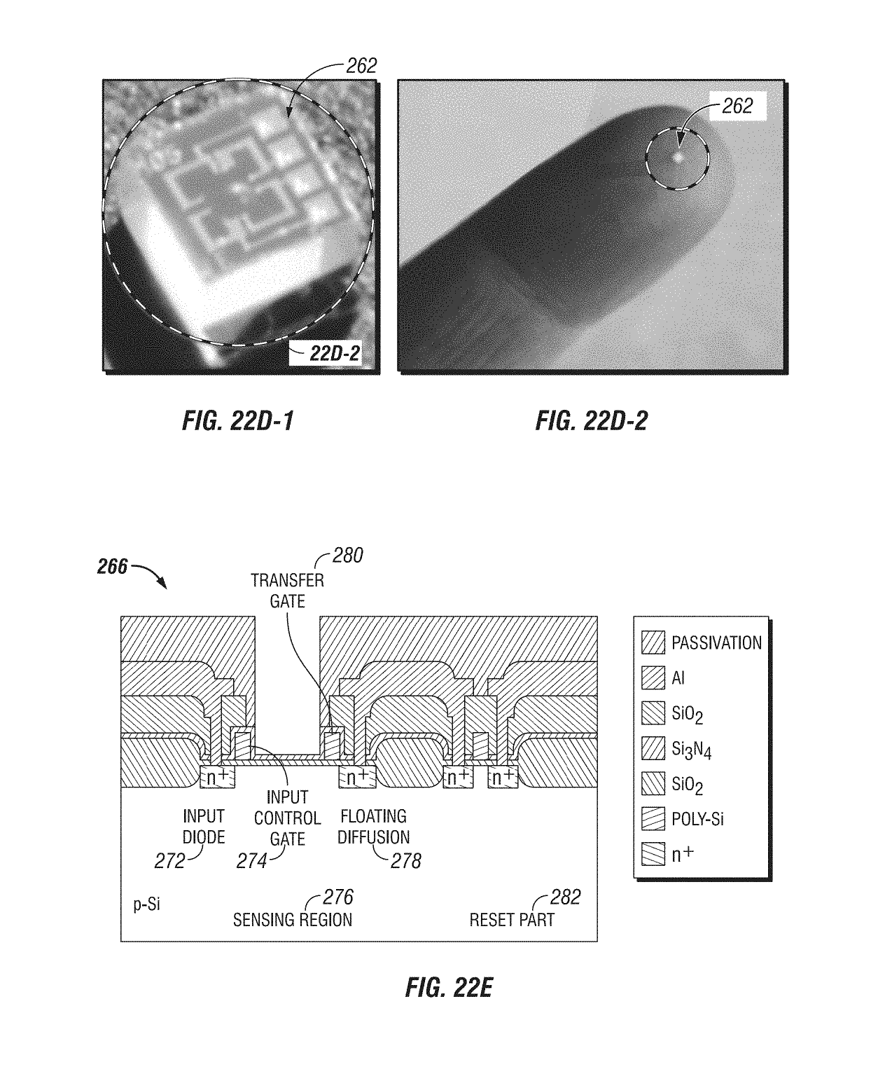

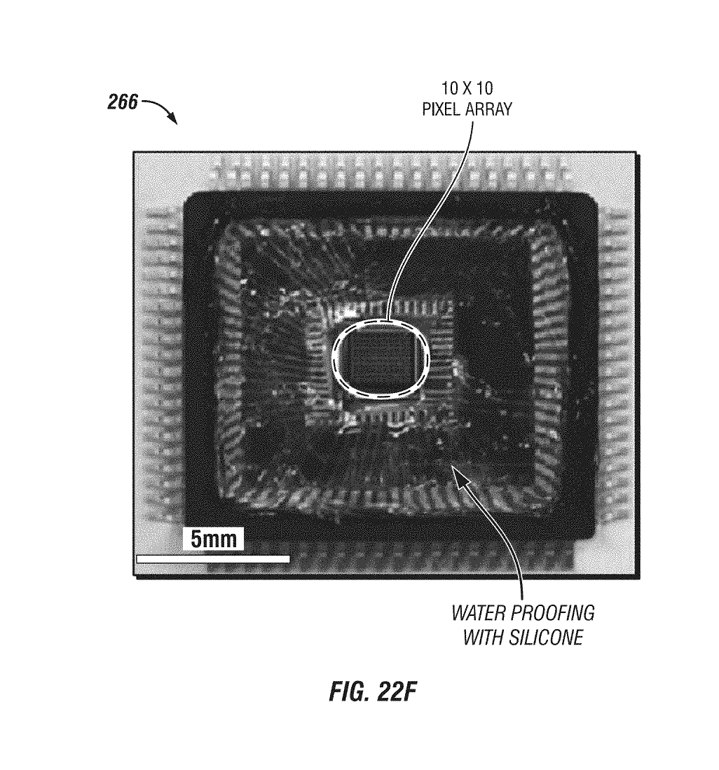

FIG. 21 still referring to the device in FIGS. 12A-12B, shows, in further detail of multiple sensor application, an embodiment where multiple types of sensors are imbedded on a single array 230 of a flexible printed circuit board 254. FIGS. 22A-F show embodiments of various types of sensors 230 that are capable of measuring, conditioning, and transmitting data for temperature 260, pressure 262, moisture/humidity 264, pH 266, and vibration frequency stimulation parameters. Application of these multiple types of sensors has been demonstrated for wound management. In some embodiments, data generated and collected from all of these sensors is conditioned and transmitted using wireless technology to a smartphone or smart device via an app Sensors 230 are miniaturized for use in the ocular region via the heating mask 140.

Temperature sensors 260 used in embodiments of this disclosure is depicted in FIG. 22A. FIG. 22B shows a performance graph of the miniaturized temperature sensing device 260. As shown in FIG. 22B, electrical resistance (in ohms) of the temperature sensor 260 corresponds with the temperature. In some embodiments, temperature sensors 260 include a printed miniature format showing connection leads for data acquisition beyond the printed circuit board 254.

Referring now to FIG. 22C, an embodiment of a moisture/humidity sensing device 264 used in the mask 140 is shown. Moisture is an important parameter considering dry eye syndrome, sensing a patient's moisture level would be helpful for the physician to address the need whether to add additional moisture to the treatment. Moisture plays an important role in determining improvement for dry eye syndrome. In some embodiments, an additional layer of an aqueous solution provided and measured by micro-sensors 264 would benefit the treatment as adequate fluid balance is achieved. A continuous moisture level measurement provides informed decisions about treatment. In some embodiments, moisture is measured by a pair of replaceable, one-time use silver chloride electrodes. After a period of about 10 to about 30 seconds, the sensor 264 measures the moisture level by recording the impedance value shown in the onboard electronics. The porous film layer 268 surrounding the sensor is bi-functional where the porous film layer 268 allows moisture to reach the sensor 264 and provides mechanical support for the sensor 264 in-situ. Data generated is then transmitted via connecting tags 270 to a smartphone or smart device for processing and display. In some embodiments, a replaceable sensor is used when recommended as part of the sensor array 230.

Referring now to FIG. 22D-1, a pressure sensor 262 is shown including a miniaturized diaphragm-based silicon pressure sensor. A Wheatstone bridge unit is incorporated using silicon-on-insulator technology. Pressure applied to the surface of the closed eyelids is a parameter for determining patient comfort and mask tightness when treating dry eye syndrome. There are a number of miniaturized pressure devices 262 being manufactured in industry and size is of concern when putting the multiple sensor array 230 into use. FIG. 22D-2 shows a photograph of a miniature pressure sensing device 262 that is powered by a small local source and provides data to a smartphone or smart device via wireless technology. On the right hand side of FIG. 22D, the miniature pressure sensing device 262 is placed on a fingertip for size comparison.

FIG. 22E shows a depiction of a miniaturized pH sensing device 266. FIG. 22F shows a photograph of the miniaturized pH sensing device 266, where the areas other than the 10×10 pixel array are waterproofed with silicone. A wide range of pH measurement approaches are available, such as pH-sensitive polymers, ion sensitive field-effect transistors, near infrared spectroscopy. In some embodiments, a two-dimensional pH image sensor is manufactured by employing a CMOS fabrication process. Referring to FIG. 22E, the pH sensing device 266 includes an input diode 272, input control gate 274, ion sensing region 276, floating diffusion region 278, transfer gate 280, reset switch 282 and a source follower circuit. Employing CMOS technology, as shown in FIG. 22E, layers of poly-Si, SiO₂, Si₃N₄, Al, and a passivation layer can be deposited and patterned on a base substrate. In some embodiments, an RFID-based wireless optical chemical pH sensor 266 with contactless power and data interface may be employed. The optical chemical pH sensor 266 is based on the differential absorbance of light in bromocresol green (BCG) in which BCG is pH sensitive.

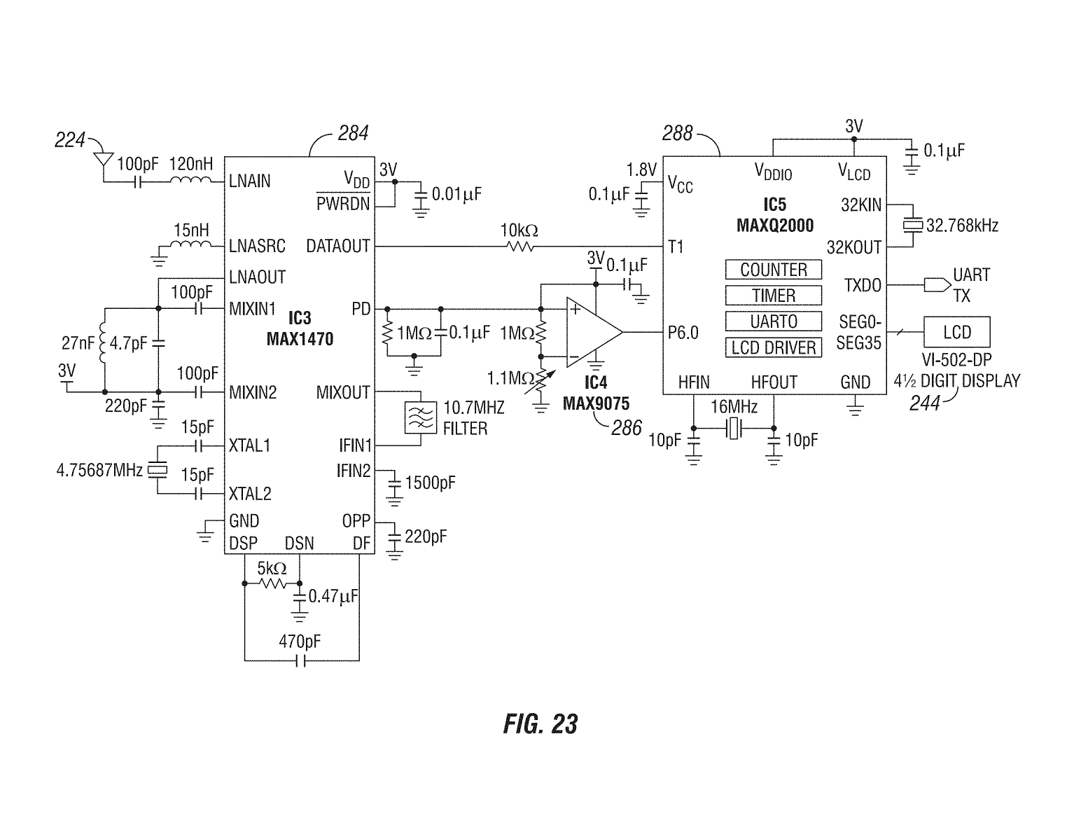

Referring to FIG. 23, an exemplary commercial wire schematic is shown for a smartphone or smart device to receive, process and display information from a wireless sensor array. Digitally-converted and wirelessly transmitted sensor data is received by IC3 284 via antenna 224. The data is further processed by IC3 284, IC4 286, and IC5 288. IC5 288 transmits the processed sensor data to the display 244 for visual display. Those skilled in the art would recognize that various means exist for a smartphone or smart device to receive, process and display information from a wireless sensor array.

According to at least one embodiment, vibration and temperature are controlled by pulse width modulation (PWM). Switching-voltage regulators employ PWM control for the switching elements. The PWM signal is either generated from a control voltage (derived from subtracting the output voltage from a reference voltage) combined with a saw tooth waveform running at the clock frequency for the voltage-mode regulator, or by adding a second loop feeding back an inductor current for current-mode control. Devices employ techniques such as voltage feed-forward for voltage-control designs and slope compensation for current-mode units.

In some embodiments, both types of topology are employed in the system. In other embodiments, component parts are linked together in the system. Voltage-mode control switching regulators are used in some embodiments when wide-input line or output-load variations are desired, under light loads (when a current-mode control-ramp slope would be too shallow for stable PWM operation), in noisy applications (when noise from the power stage would find its way into the current-mode control feedback loop), and when multiple-output voltages are needed with good cross regulation.

In some embodiments, current-mode control devices are used for applications where the supply output is high current or very-high voltage; the fastest dynamic response is sought at a particular frequency, input-voltage variations are constrained, and in applications where cost and number of components must be minimized as in the innovations stated here within.

According to at least one embodiment, the reusable mask 140 and even the entire system is suitable for mobile control, in which the device is easily hand held and carried for patient use. Control may also be driven by a smartphone or smart device using operating systems such as iOS, Android or Windows mobile, or other similar interfaces. Mobile medical interfaces are used in products such as a Zebra MC40 Mobile Computer. Similar platforms, or other Wi-Fi, cell phone or Bluetooth connected interfaces can be used to control the patient's first in-office use of the system. In some embodiments, a range of frequencies are tested and output data from the sensors is stored. The data storage and its associated algorithm may determine the best treatment mode for following office visits or transfer an optimal program to an at-home unit. These mobile interfaces further create efficiencies for the office staff by automatically storing patient records to the electronic medical records (EMR) of the first and subsequent uses. These records include patient name, time and date of use, frequencies explored and sensors output during that time. The at-home unit would also serve as a record of patient compliance to prescribed therapy.

According to at least one embodiment, the harmonic resonance heating mask 140 is preferentially supplied as a kit. Kits include one or more devices, and varying numbers of replacement heaters depending on kit size. Kits may include both elements of the one-time use components and reusable components. In some embodiments, for example, a kit might include the one-time use heating element 100, the reusable miniature harmonic resonance frequency stimulation generator 130 pairs that fit into the eye patch component and plug into a USB 134 port and the one-time use coupling device. Kits may be provided to a patient during an office visit as the equipment used to define the correct resonance frequency stimulation would be available in the practitioner's office. Commercial kits may also be provided with very specific frequencies and then purchased directly by an informed customer.

According to at least one embodiment, as mentioned in describing the mobile controller, after a patient's first use of the system in the physician's office, the patient may be prescribed to continue therapy on a more frequent basis at home. As an alternate embodiment, this system could be simplified for the home user. This system would have a reusable mask 140 with single use disposable, or reusable built-in heating elements 100 and resonance frequency stimulation generators 130, accommodate an optional disposable coupling device and come with appropriate power supply and control, including a mobile and wirelessly connected controller. The at-home monitoring system would not require a full range of vibration frequencies as the optimal frequency stimulation and pattern was determined in the original office use and that pattern is programmed into the individual user's system. Similarly, the full sensing capability is not needed for home use. A cell phone, Wi-Fi or Bluetooth connected controller may also create a record of use for the patient's EMR. In some embodiments, patterns of noncompliance or misuse may create an alert to go directly to the patient and/or back to the treating physician.

A further alternate embodiment may include a system that employs single use heating discs 100. This could be used for either the office based or home use products. The disposable heating disc 100, being hand moldable to conform to an individual's anatomy, would fit into the pocket in the heating mask 140. This heating disc 100 element could also be built into and supplied as part of the coupling device that contacts the skin and includes a combined single disposable item. As shown in FIGS. 6A and B, in some embodiments, a moldable heating disc 100 with a low cost RFSVG 130 may be located in the disposable heating disc 100 and connected via the USB 134 port for control and energy. A reusable RFSVG 130 (mini-vibrator) can be placed in the manufactured pocket location located in the moldable heating disc 100. This approach may be used for both the moldable gel and polymer eye piece.

According to at least one embodiment, there is provided a method of treating dry eye disease or MGD. These methods include the initial physician's office based use where optimal treatment parameters are determined and then stored for later use either in subsequent office visits or home use.

The advantages of the devices disclosed include, without limitation, that it is portable, easy to transport, reliably functions as intended, and is simple and convenient to activate and use. Another advantage is that it is easy to integrate these devices into a reusable face mask or eye patch because they are relatively small and lightweight, showing the parallel utility of the device components stated herein.

A further alternate embodiment may include an integrated real-time imaging device to detect optimal tuning of the RFSVG 130 to the particular patient eyelid and Meibomian glands. In some embodiments, for example, optoacoustic imaging or photoacoustic imaging is insensitive to photon scattering within biological tissue and, unlike conventional optical imaging methods, makes high-resolution optical visualization deep within tissue possible. A key empowering feature is the development of video-rate multispectral imaging in two and three dimensions, which offers fast spectral differentiation of distinct photo-absorbing moieties. In some embodiments, the imaging device provides a real-time-image-based assessment of the optimal settings for the miniature RFSVG 130 at which there is maximal movement of the eyelids, Meibomian glands, and lipid fluid within the Meibomian glands.

According to at least one embodiment, there is provided means for providing a physician and a patient with a metric related to the state of the dry eye disease being treated. This metric will correlate to the severity of disease, and may be measured and provided both before and after treatment. Increased sensitivity to light is a well-known proxy for severity of dry eye disease. According to at least one embodiment, there is provided a light sensor configured to measure light sensitivity of the eye being treated and to provide a subjective light sensitivity score as a diagnostic indicator.

According to at least one embodiment, there is provided a method in which prior to initiating a treatment, the patient looks at a target in the mask 140 or at a distance. A light-emitting diode (LED) with a controllable spectrum is mounted to a head-mounted mask. The LED in the mask will turn on at an adjustable initial setting. The patient adjusts the intensity to the maximum comfortable level, with a physical rheostat or other controller. Right and left eyes may be tested sequentially, or both eyes may be tested simultaneously. The light intensity setting is recorded electronically. At the end of the treatment, the patient is exposed to light and the light sensitivity measurement is performed again. Each time the patient uses the device, their pre-treatment and post-treatment light sensitivity is recorded electronically, and comparison made with the previous light sensitivity scores. The comparison provides an indicator of treatment success, as well as dry eye disease stability, improvement, or worsening. According to at least one embodiment, the system can include a feature to automatically increase or decrease the treatment duration and/or intensity based on the light sensitivity measure, and relative change from the previous light sensitivity value.

According to at least one embodiment, the heating mask 140 can be configured to fit a single eye. The single-eyed heating mask 140 can be configured to fit either the patient's right or left eye. In some embodiments, the heating mask 140 can include two single-eyed heating masks, one configured to the right eye and the other configured to the left eye.

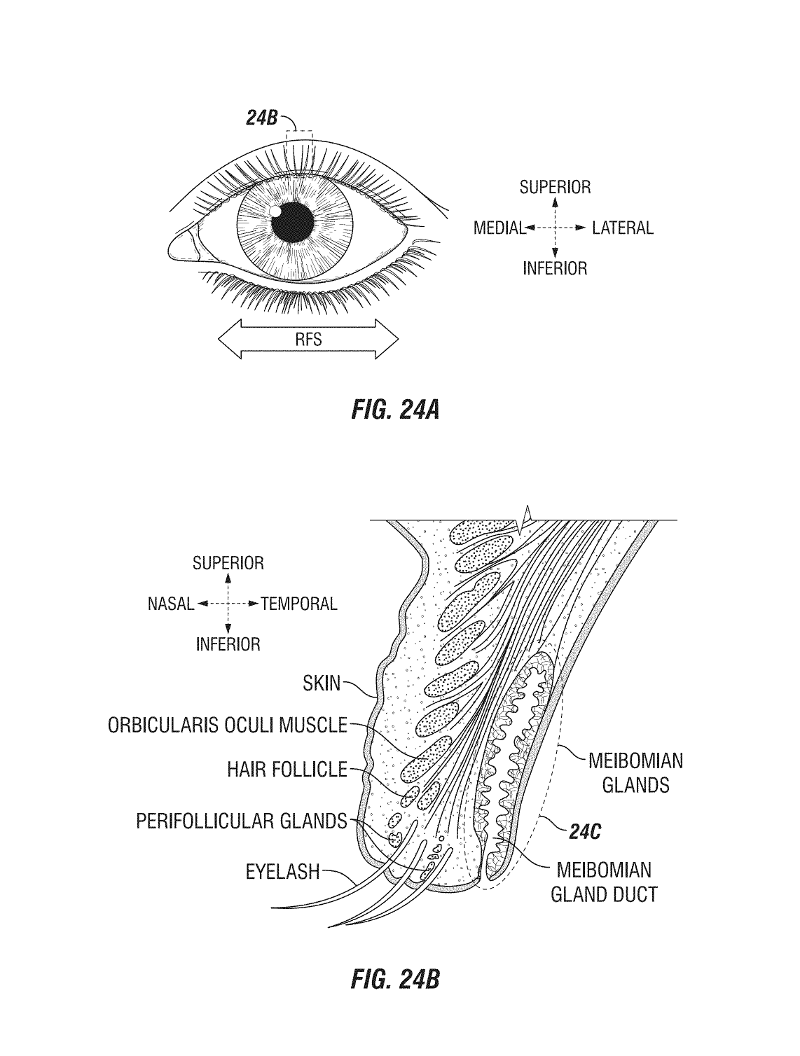

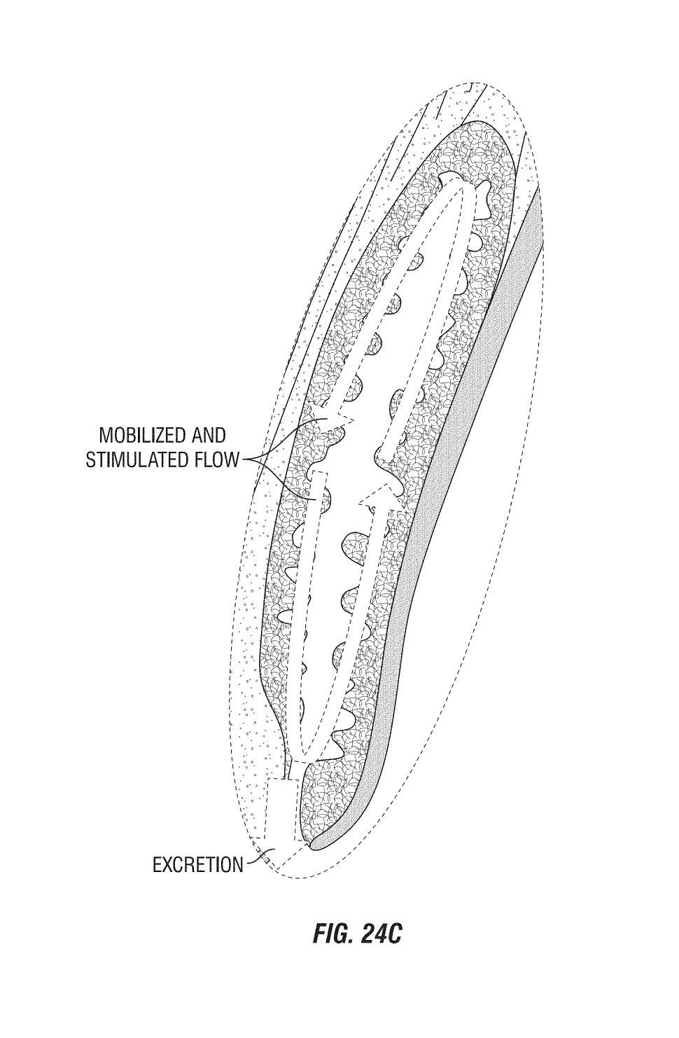

FIG. 24A shows a front view of a human eye. FIG. 24B shows an enlarged cross-sectional view of an upper eyelid of the human eye. FIG. 24C shows a cross-sectional view through a Meibomian gland of an upper eyelid of a human eye where the harmonic resonance frequency stimulation vibration mobilizes and stimulates flow of the Meibum lipids within a Meibomian gland. The mobilized and stimulated flow leads to enhanced excretion of the Meibum lipids.

According to at least one embodiment, the harmonic resonance frequency stimulation is a vector force. The harmonic resonance frequency stimulation exhibits traits substantially similar to a longitudinal wave. In some embodiments, the harmonic resonance frequency stimulation exhibits traits substantially similar to a longitudinal standing wave.

According to at least one embodiment, the primary axis of vibration is substantially parallel to the medial-lateral axis, as shown in FIG. 24A. The primary axis is tangential to the depth of the Meibomian gland duct, which is shown as the superior-inferior axis in FIGS. 24A and 24B. As shown in FIG. 24B, the primary axis of vibration is substantially tangential to the opening of the Meibomian gland duct. In some embodiments, the amplitude of the vibration, corresponding to the vector force, is configured to be substantially parallel to the medial-lateral axis, as shown in FIG. 24A. Accordingly, a RFSVG 130 may generate harmonic resonance frequency stimulation vibrating in the medial-lateral direction, which is substantially tangential to the depth of the Meibomian gland duct.

According to at least one embodiment, the primary axis of vibration can be substantially parallel to the superior-inferior axis, as shown in FIGS. 24A and 24B. The amplitude of the vibration, corresponding to the vector force, is configured to be substantially parallel to the superior-inferior axis, as shown in FIGS. 24A and 24B. Accordingly, a RFSVG 130 may generate harmonic resonance frequency stimulation vibrating in the superior-inferior direction, which is substantially parallel to the depth of the Meibomian gland duct.

According to at least one embodiment, the harmonic resonance frequency stimulation can be a superposition of two or more longitudinal vibrational waves. In some embodiments, the harmonic resonance frequency stimulation is a superposition of two longitudinal vibrational waves, where the primary axis of the first vibration can be substantially parallel to the superior-inferior axis while the primary axis of the second vibration can be substantially parallel to the medial-lateral axis, both axes as shown in FIGS. 24A and 24B. The amplitude of the first vibration, corresponding to the first vector force, is configured to be substantially parallel to the superior-inferior axis, as shown in FIGS. 24A and 24B. The amplitude of the second vibration, corresponding to the second vector force, is configured to be substantially parallel to the medial-lateral axis, as shown in FIG. 24A. Accordingly, a RFSVG 130 may generate two simultaneous harmonic resonance frequency stimulations, one vibrating in the superior-inferior direction, which is substantially parallel to the depth of the Meibomian gland duct, and the other vibrating in the medial-lateral direction, which is substantially tangential to the depth of the Meibomian gland duct.

According to at least one embodiment, the direction of the harmonic resonance frequency stimulation vector force can be selected by the device operator based on the degree of MGD (i.e., truncated gland ducts, clogged or plugged gland orifices versus open gland orifices).

According to at least one embodiment, the harmonic resonance frequency stimulation mobilizes the Meibum lipids within the Meibomian glands. The mobilization is achieved by inducing shear forces using vibration at the resonance frequency or frequencies of the patient's eyelid and Meibomian gland complex. As shown in FIG. 24C, a flow of the mobilized Meibum lipids is generated as a result of the vibration. After the Meibum lipids are mobilized, natural eye blinking will lead to the excretion of these lipids.

According to at least one embodiment, the reusable mask can include a bladder-type coupling device to provide substantially full contact to the patient's individual eyelid and periorbital three-dimensional anatomy and surface topography. The bladder-type coupling device can be filled with gaseous or fluidic medium, or foam. Hydraulics or pneumatics can be applied to control the coupling device. In some embodiments, a hydraulic medium is used in the bladder-type coupling device, where the hydraulic medium has a viscosity suitable for conforming to the patient's individual eyelid and periorbital three-dimensional anatomy and surface topography. The hydraulic medium is suitable for transmitting harmonic resonance frequency stimulation vibration generated by the RFSVG 130 to the patient's Meibomian glands. The hydraulic medium is suitable for transmitting the vibration in any direction. In some embodiments, the bladder-type coupling device includes channels to provide direction control of the vibration. In some embodiments, temperature and pressure control of the reusable mask can achieved by hydraulically inserting the hydraulic medium into the bladder-type coupling device.

According to at least one embodiment, the heating mask 140 is operable to change configuration of applying heat and the resonance frequency stimulation vibration. In some embodiments, the heating mask 140 is operated such that heat (for example, provided by the heating disc 100) and the resonance frequency stimulation vibration (for example, provided by the RFSVG 130) are applied to the patient's eyelid area sequentially and alternately, but not simultaneously. The heating mask 140 can be internally or externally programmed to achieve this sequence. In some embodiments, the medical practitioner cannot override the programmed sequence. In other embodiments, explicit instructions, such as an instruction manual, can be given to the medical practitioner to operate the heating mask 140 by applying heat and the resonance frequency stimulation vibration sequentially and alternately.

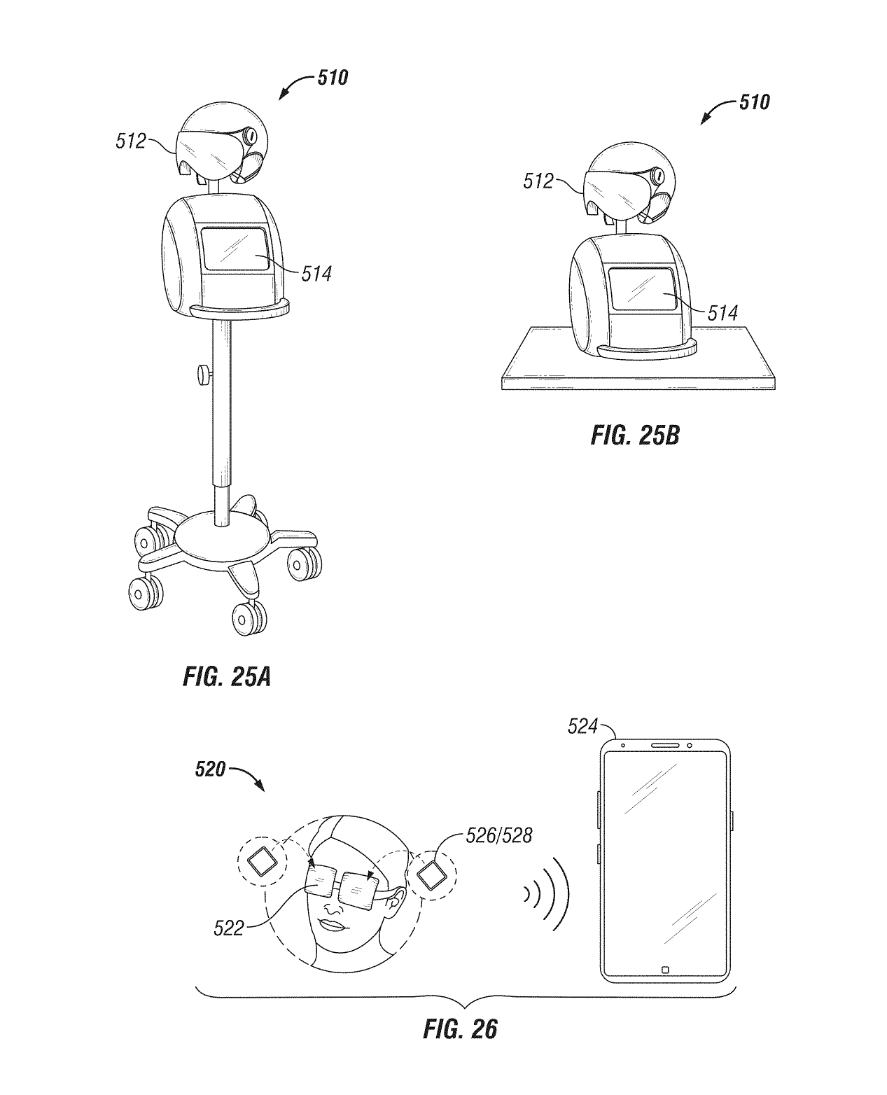

Referring now to FIGS. 25A and B, two embodiments of an in-office ophthalmic eyelid treatment monitoring system 510 is shown. In some embodiments, the in-office monitoring system 510 is configured to be used in the medical practitioner's office for treatment of patients. In some embodiments, the in-office monitoring system 510 provides heat and resonance frequency stimulation vibration to the patient's eyelids to enhance flow of lipids from the Meibomian glands. The in-office monitoring system 510 may either be mobile or stationary; the embodiment shown in FIG. 25A is a mobile unit and the embodiment shown in FIG. 25B is a counter top unit.

Still referring to FIGS. 25A and B, the in-office monitoring system 510 includes an adjustable mask 512 to comfortably fit any ocular anatomy for personalized treatment. The mask 512 is reusable and delivers personalized resonance frequency stimulation vibratory energy and heat. In some embodiments, the mask 512 includes a single use disposable, moldable coupling device or gel or hydrogel which conducts heat and vibratory energy to the eyelids. The coupling device or gel or hydrogel molds to the patient's individually unique eyelid and periorbital three dimensional anatomy and surface topography. In effect, the coupling device or gel or hydrogel provides customized personal tuning of resonance frequency stimulation to patient's eyelids and Meibomian glands. Optionally, the monitoring system 510 may provide music to the patient while receiving therapy to enhance relaxation.

Still referring to FIGS. 25A and B, the in-office monitoring system 510 also includes a monitor/controller 514 with a display, optionally an LCD display, that shows tuning parameters such as optimized and personalized temperature and vibration resonance frequency stimulation. Optionally, the monitoring system 510 includes storage space for single use coupling device or gel or hydrogel that conforms to the patient's individually unique eyelid and periorbital three dimensional anatomy and surface topography.

Referring now to FIG. 26, an embodiment of an at-home ophthalmic eyelid treatment monitoring system 520 is shown. In some embodiments, the at-home monitoring system 520 is configured to be used individually by patients for continued treatment. In some embodiments, the at-home monitoring system 520 provides heat and optionally includes resonance frequency stimulation vibration to the patient's eyelids to enhance flow of lipids from the Meibomian glands.

Still referring to FIG. 26, the at-home monitoring system 520 may include an adjustable mask 522 to comfortably fit any eyelid and periorbital three dimensional anatomy and surface topography for personalized treatment. In some embodiments, the mask 522 is reusable and provides heat and optionally delivers personalized resonance frequency stimulation vibratory energy and heat. In some embodiments, the mask 522 may include a single use disposable, moldable coupling gel which conducts heat and vibratory energy to the eyelids. The coupling gel molds to the patient's individually unique eyelid and periorbital three dimensional anatomy and surface topography. In effect, the coupling gel provides customized personal tuning of resonance frequency stimulation vibration to the patient's eyelids and Meibomian glands. Optionally, the coupling gel may be of multiple use, since the mask 522 is already personalized to the patient's facial anatomy.

Still referring to FIG. 26, the at-home monitoring system 520 may utilize a smartphone or smart device 524 for control and display purposes. Tuning parameters such as optimized and personalized temperature and vibration resonance frequency stimulation vibration are shown on a smartphone or smart device 524 via an App. In some embodiments, the mask 522, including sensors 526 and energy sources 528 of the mask 522, and the smartphone or smart device 524 may be connected wirelessly. Imbedded sensors 526 detect and transmit usage date to a smartphone's or smart device's dry eye management app. In some embodiments, the mask 522 may include electronic components shown in FIGS. 14 and 15. In some embodiments, the smartphone or smart device 524 may include electronic components shown in FIGS. 16 and 23.

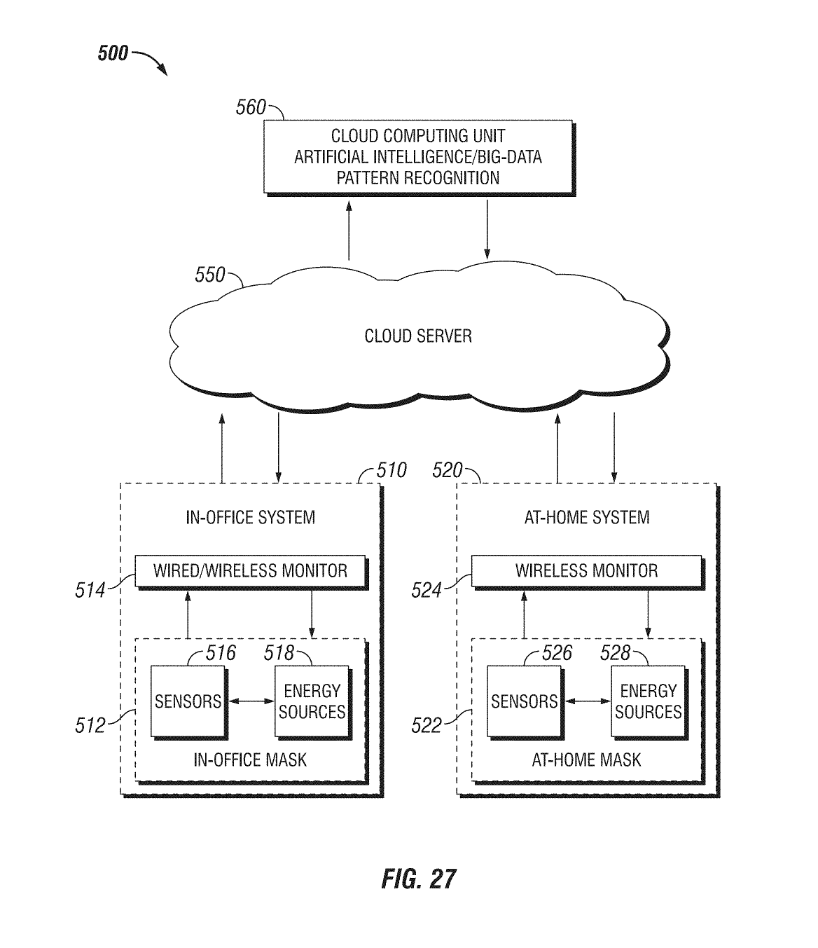

Referring now to FIG. 27, an embodiment of a cloud-based ophthalmic eyelid treatment monitoring system 500 is shown. A cloud server 550 receives various forms of data from both an in-office monitoring system 510 and an at-home monitoring system 520. Data is transmitted to the cloud server 550 from the monitoring systems 510, 520 and vice versa via wired or wireless communication. Wireless communication may occur via a smartphone or smart device using wireless protocols, such as Bluetooth, Wi-Fi, cellular network, or the like, to transmit data to the cloud server 550. The cloud server 550 utilizes various forms of algorithms used in cloud computing to determine optimal tuning parameters, such as temperature and resonance vibration frequency stimulation, to be transmitted to the in-office mask 512 and at-home mask 522. According to at least one embodiment, the cloud server 550 transmits data to and receives data from an external cloud computing unit 560 that utilizes artificial intelligence and/or big-data pattern recognition. In other embodiments, the cloud computing unit 560 utilizing artificial intelligence and/or big-data pattern recognition is internally integrated into the cloud server 550.

Still referring to FIG. 27, the in-office system 510 is located in the medical practitioner's office and may be remote from the cloud server 550. As described in FIG. 25, the in-office system 510 includes a monitor 514 and a mask 512. The in-office mask 512 includes various types of sensors 516 configured to generate data responsive to a vibration and heating profile of the patient's eyelid and periorbital three dimensional anatomy and surface topography. The in-office monitor 514 receives data generated from the imbedded sensors 516 located in the mask 512 unit via wired or wireless communication. In some embodiments, the in-office monitor 514 is connected wirelessly with both the in-office mask 512 and the cloud server 550. Via a smartphone or smart device app, the medical practitioner may access the in-office monitor 514 to prescribe heat and vibration profiles for the energy sources 518 based on the optimal tuning parameters provided by the cloud server 550. In some embodiments, the data generated from the imbedded sensors 516 are raw data. In some embodiments, by accessing the in-office monitor 514, the medical practitioner provides data derived from integrated light sensitivity metric or data derived from patient questionnaires. In some embodiments, the in-office monitor 514 is configured to access the patient's smartphone or smart device. In some embodiments, the in-office monitor 514 is configured to retrieve any metadata related to the patient. The in-office monitor 514 then transmits data received from the sensors 516 to the cloud server 550. In some embodiments, data transmitted from the in-office monitor 514 to the cloud server 550 is encrypted. Calculated optimal tuning parameters derived from the cloud server 550 are transmitted to the in-office monitor 514 for display. In some embodiments, optimal tuning parameters transmitted from the cloud server 550 to the in-office monitor 514 are encrypted. Optionally, the in-office monitor 514 may further process or store the optimal tuning parameters received from the cloud server 550. Optionally, the in-office monitor 514 is configured to self-derive optimal tuning parameters. According to at least one embodiment, the in-office monitor 514 transmits optimal tuning parameters to imbedded energy sources 518 located in the mask 512 via wired or wireless communication. Energy sources 518 include sources configured to convey thermal and vibratory energy to the patient's eyelid and surrounding anatomy. The energy sources 518, upon receiving the optimal tuning parameters from the in-office monitor 514, releases thermal and vibratory energy pursuant to the optimal temperatures and optimal resonance vibration frequencies. Optionally, the energy sources 518 are configured to receive tuning parameters directly from the sensors 516 via wired or wireless communication.

Still referring to FIG. 27, the at-home system 520 is located anywhere out of the medical practitioner's office and also may be remote from the cloud server 550. As described in FIG. 22, the at-home system includes a monitor 524 and a mask 522. The at-home mask 522 includes various types of sensors 526 configured to generate data responsive to a vibration and heating profile of the patient's eyelid and periorbital three dimensional anatomy and surface topography. The at-home monitor 524 receives data generated from the imbedded sensors 526 located in the mask 522 unit via wireless communication. In some embodiments, the at-home monitor 524 is connected wirelessly with both the mask 522 and the cloud server 550. Via a smartphone or smart device app, the medical practitioner may access the at-home monitor 524 to prescribe heat and vibration profiles for the energy sources 528 based on the optimal tuning parameters provided by the cloud server 550. In some embodiments, the medical practitioner may access the in-office monitor 514, either by wired or wireless communication, to prescribe heat and vibration profiles for the at-home energy sources 528. In some embodiments, the medical practitioner may access the at-home monitor 524 wirelessly to prescribe heat and vibration profiles for the in-office energy sources 518. In some embodiments, the data generated from the imbedded sensors 526 are raw data. In some embodiments, by accessing the at-home monitor 524, the medical practitioner provides data derived from integrated light sensitivity metric or data derived from patient questionnaires. In some embodiments, the at-home monitor 524 is configured to access the patient's smartphone or smart device. In some embodiments, the smartphone or smart device and/or the app installed therein is the at-home monitor 524. In some embodiments, the at-home monitor 524 is configured to retrieve any metadata related to the patient. The at-home monitor 524 then transmits data received from the sensors 526 to the cloud server 550. In some embodiments, data transmitted from the at-home monitor 524 to the cloud server 550 is encrypted. Calculated optimal tuning parameters derived from the cloud server 550 are transmitted to the at-home monitor 524 for display. In some embodiments, optimal tuning parameters transmitted from the cloud server 550 to the at-home monitor 524 are encrypted. Optionally, the at-home monitor 524 may further process or store the optimal tuning parameters received from the cloud server 550. Optionally, the at-home monitor 524 is configured to self-derive optimal tuning parameters. According to at least one embodiment, the at-home monitor 524 transmits optimal tuning parameters to imbedded energy sources 528 located in the at-home mask 522 via wireless communication. Energy sources 528 include sources configured to convey thermal and vibratory energy to the patient's eyelid and surrounding anatomy. The energy sources 528, upon receiving the optimal tuning parameters from the at-home monitor 524, releases thermal and vibratory energy pursuant to the optimal temperatures and optimal resonance vibration frequencies. Optionally, the energy sources 528 are configured to receive tuning parameters directly from the sensors 526 via wired or wireless communication.

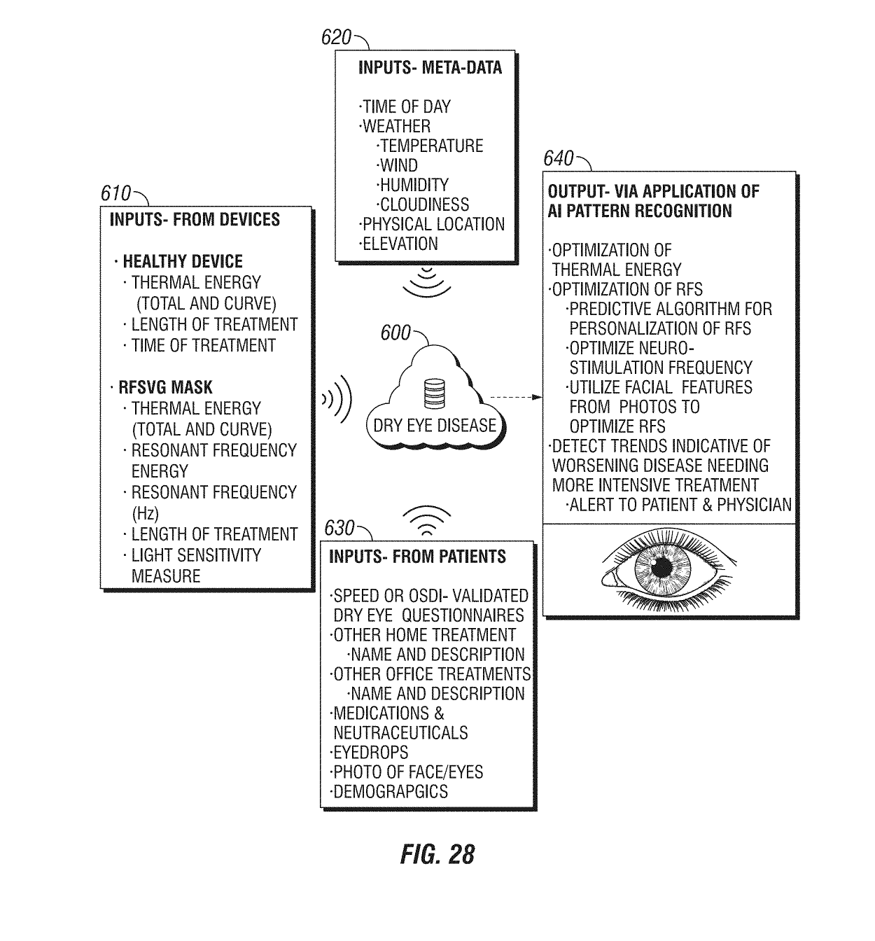

Referring now to FIG. 28, a schematic of a dry eye disease smart cloud database 600 is shown, depicting examples of inputs and outputs. In some embodiments, device inputs 610 are transmitted, wired or wirelessly, to the smart cloud database 600 from masks 512, 522 containing eyelid heating devices and/or the RFSVG. Certain eyelid heating devices may transmit certain treatment data such as thermal energy (total and curve), treatment length, and treatment time. The RFSVG-embedded mask may transmit certain treatment data such as thermal energy (total and curve), resonance frequency energy, resonance frequency (in Hz), treatment length, and light sensitivity measurements. In some embodiments, meta-data inputs 620 are transmitted, wired or wirelessly, to the smart cloud database 600. The meta-data inputs 620 may include time of day, weather parameters such as temperature, wind, humidity, and cloudiness, physical location, and elevation. In some embodiments, patient inputs 630 are transmitted, wired or wirelessly, to the smart cloud database. Patient inputs 630 may include validated dry eye questionnaires such as SPEED or OSDI. Patient inputs 630 may include other home treatments which include name and description or other office treatments which include name and description. Patient inputs 630 may include medications and neutraceuticals, eyedrops, photographs of the patient's face or eyes, and patient demographics.

Still referring to FIG. 28, in some embodiments, once the inputs 610, 620, 630 are transmitted to the smart cloud database 600, the database 600 undergoes data analysis to generate outputs 640 via application of artificial intelligence/big-data pattern recognition. The outputs 640 may include optimization of thermal energy. The outputs 640 may include optimization of resonance frequency stimulation (RFS), which involves predictive algorithms for personalization of RFS, optimization of neuro-stimulation frequencies, and utilization of facial features from photographs to optimize RFS. The outputs may also include detecting trends indicative of worsening disease that may require more intensive treatment, which involves alerts to the patient and the medical practitioner. In some embodiments, the cloud server 550 and/or the cloud computing unit 560 shown in FIG. 27 may include the smart cloud database 600.

According to at least one embodiment, the data stream is suitable for interpreting patient compliance with respect to treatment. The cloud database 600 is compliant with the Health Insurance Portability and Accountability Act (HIPAA), available through an electronic medical record system, where the medical record system includes subjective patient-entered data and is available to the patient.

According to at least one embodiment, there is provided a cloud-based, dry eye disease database 600 that includes patient demographics, treatments, and outcomes. The cloud-based database 600 is HIPAA compliant. Values of the database 600 is derived through application of pattern recognition, artificial intelligence, and big-data analytics to improve treatments and outcomes for dry eye disease and MGD patients.

According to at least one embodiment, sensors 516, 526 are imbedded in the mask 512, 522 for optimization of dry eye disease treatment. Artificial intelligence and big-data pattern recognition may be utilized to accomplish new insights into optimal treatment based on individual patient characteristics.

According to at least one embodiment, the mask 512, 522 including its imbedded components generates data to be transferred wirelessly to a smartphone or smart device with a Smart App running while monitoring usage. Data is stored in a cloud database 600, which is HIPAA compliant.

According to at least one embodiment, various sources of data 610, 620, 630 are incorporated into the cloud database 600. Data is recorded for each use of the mask 512, 522, including usages at home and in the physician's office.

According to at least one embodiment, data input 610 includes data derived from the embedded sensors 516, 526 of the mask 512, 522. Embedded sensors 516, 526 may collect data relating to the total thermal energy delivered to the patient's eyelid, including data relating to the length of treatment and temperature curves. Embedded sensors may collect data relating to the total resonance frequency stimulation (RFS) energy delivered to the patient's eyelid and periorbital three dimensional anatomy and surface topography, including data relating to the vibratory mechanical energy curve derived throughout treatment. Embedded sensors 516, 526 may collect data relating to the RFS frequency delivered to the patient's eyelid and periorbital three dimensional anatomy and surface topography, including data relating to the frequency stimulation curve derived throughout treatment. Embedded sensors 516, 526 may also collect data relating to the external wind or air movement, data relating to the blink rate of the patient, and data related to the patient's length of sleep time.

According to at least one embodiment, data input 610 includes data derived from integrated light sensitivity metric. This metric is a measure of subjective sensitivity to light, which correlates to the severity of the disease, and optionally may be measured and be provided to the medical practitioner and patient both before and after treatment. Increased sensitivity to light is a well-known proxy for the severity of dry eye disease. A light sensitivity measurement includes a subjective light sensitivity score as a diagnostic indicator.

According to at least one embodiment, data input 630 includes data derived from patient questionnaires. Questionnaires may be related to patient demographics, including identity, age, and sex. Data derived from questionnaires may be related to patient responses to standardized and validated dry eye symptom questionnaires such as Ocular Surface Disease (OSD) and Standardized Patient Evaluation of Eye Dryness (SPEED) questionnaires. Questionnaires may be related to other dry eye treatments received in the past such as drops, intense pulsed light, and thermal pulsation. Questionnaires may be related to the patient's current or past medications, vitamins, and nutraceuticals.

According to at least one embodiment, data input 620 includes data derived from recorded metadata. Metadata may include information related to date, time of day, location, humidity, and ambient temperature.

According to at least one embodiment, data input 630 includes data derived from a smart phone or a smart device. As a non-limiting example, information related to the patient's eyes may be derived from a patient's facial photograph stored in the smart phone or smart device.

According to at least one embodiment, there is provided applications of artificial intelligence and big-data pattern recognition. Utilizing artificial intelligence and big-data pattern recognition provides optimization of thermal energy treatment to improve symptoms by generating an optimized temperature profile. Utilizing artificial intelligence and big-data pattern recognition provides optimization of RFS with respect to frequency stimulation and magnitude by analyzing the patient's demographics, photographs, and outcomes. According to at least one embodiment, optimal frequency stimulation is predicted for stimulation of the Meibomian glands. According to at least one embodiment, optimal frequency stimulation is predicted for neuro-stimulation of the Trigeminal cranial nerve, and optionally the ophthalmic branch of the Trigeminal cranial nerve, to disrupt central pain syndrome mediated through the Trigeminal Ganglion and/or hypothalamus in patients with a central pain component, which is a well-known feature of the disease in patients with dry eye disease.

According to at least one embodiment, utilizing artificial intelligence and big-data pattern recognition provides predictive algorithms based on photographic anatomic features of eyelids and the face to personalize treatment parameters, including frequency stimulation and magnitude of RFS, and thermal energy delivered. Utilizing artificial intelligence and big-data pattern recognition provides predictive algorithms to recommend optimal time and number of treatments. Utilizing artificial intelligence and big-data pattern recognition provides identification of trends indicative of when a patient requires more intensive intervention available in their physician's office. Utilizing artificial intelligence and big-data pattern recognition provides automatic notifications sent to the physician and the patient. A dashboard view of database is provided to extract insights into patient demographics and optimized treatments.

According to at least one embodiment, end point monitors may include ultrasound techniques. The ultrasound techniques may have an ultrahigh resolution. Utilizing ultrasound imaging and analyzing images produced by ultrasound imaging provides non-invasive assessment of the viscosity of the lipids within the Meibomian glands. Other modalities may be utilized to achieve the same end. The measurement of the lipid viscosity may serve as an endpoint of treatment.

According to at least one embodiment, there is provided a monitoring system to manage dry eyes. A dry eye therapy ecosystem is built by utilizing both office treatment and home maintenance treatment. In the physician's office, medical practitioners may utilize an integrated in-office ophthalmic eyelid treatment monitoring system 510. At home, the patient may utilize an integrated, moldable warming device 522. The patient may use the device pursuant to the prescribed heat and vibration (at a resonance frequency stimulation) levels determined in the medical practitioner's office.

According to at least one embodiment, the monitoring system provides eyelid resonance frequency stimulation analysis including mechanisms to deliver eyelid resonance frequency stimulation vibration. Resonance frequency stimulation vibration may be delivered to the patient's eyelid incorporating microfluidics with vibratory motion, mechanical motor motion, and piezoelectric ultrasound vibration. The eyelid resonance frequency stimulation analysis also includes detection technology for customization of treatment. Various detection technologies may enable the system to monitor tissue response by sweeping a range of frequencies. In some embodiments, a piezoelectric crystal in receiving mode may detect a maximal or a multiple of maximal tissue vibratory responses.

According to at least one embodiment, the monitoring system provides various mechanisms of eyelid warming. Mechanisms include a warm liquid bath incorporating microfluidic devices accompanied with electrical resistive heating or other heating means so to heat the liquid in the microfluidic channels. Various liquids having various viscosities may be used in the microfluidic channels that conform to the eyelid and surrounding tissue of the patient's three dimensional anatomy. Mechanisms include direct warming such as heating means utilizing electrical resistive heating circuits, radiofrequency, microwave, and ultrasound.

According to at least one embodiment, the monitoring system provides a coupling agent that molds to the patient's eyelid and periorbital three dimensional anatomy and surface topography. The coupling agent may be a consumable component. The coupling agent transmits both warmth and vibration.

According to at least one embodiment, the coupling agent may include a microfluidic liquid interface having various viscosities, such as using hydrogel or an air- or gas-filled bladder. The coupling agent may be of single use. The coupling agent conforms to the patient's eyelid and periorbital three dimensional anatomy and surface topography, transmits thermal energy, and transmits bi-directionally vibratory energy.

According to at least one embodiment, the monitoring system 510, 520 includes a mask 512, 522. The mask 512, 522 adjusts to facial features such as the eyelid and periorbital three dimensional anatomy and surface topography. The mask 512, 522 is large enough to allow neural stimulation of the periorbital facial cranial nerves involved in the afferent component of dry eyes experienced as a central pain syndrome. The neural stimulation serves to disrupt the central pain syndrome. The mask 512, 522 is large enough to increase vascular perfusion to the periorbital region, thereby reinforcing the device's warming effect with natural warmth of tissue blood flow. The mask 512, 522 includes a conforming element over the patient's nose to enhance three-dimensional contact with the patient's eyelids, to ensure adequate conductive heating is achieved and all Meibomian glands, whether nasally, centrally, or temporally located, are warmed adequately. The mask 512, 522 reduces air gaps to avoid convective heating, which is less efficient and inaccurate. In some embodiments, shape-memory materials such as Nitinol or other suitable polymers are integrated into a nose piece, customized to the individually unique three-dimensional anatomy of the patient's nasal bridge.

According to at least one embodiment, the monitoring system 510, 520 provides a controller software. The software may be controlled or programmed by the medical practitioner in the office directly, or via a smartphone or smart device app. The software may be controlled or programmed by the medical practitioner remotely via a smartphone or smart device app.

According to at least one embodiment, the monitoring system 510, 520 provides electronic components. The electronic components control thermal energy that is transmitted to the patient and sense the patient's eyelid surface temperature. The electronic components control vibratory energy that is transmitted to the patient and sense eyelid responses to the transmitted vibration.

According to at least one embodiment, the monitoring system 510, 520 provides remote connectivity via a smartphone or smart device app. The app is configured to track symptoms and treatments. The app detects and senses remotely the time and date when treatments are delivered and the length of the treatments. The app is compatible with in-office treatments, home maintenance treatments, and other home prescribed treatments. The app is configured to inform and educate both the medical practitioner and the patient. The app is configured to communicate remotely with the medical practitioner. The app is configured to provide patient access and authorized medical practitioner access. The app maintains HIPAA compliant aggregated cloud-based database 600.

According to at least one embodiment, the mask 512, 522 is provided with embedded sensors 516, 526 and energy sources 518, 528 that communicate directly and remotely with the app, receive programming from the medical practitioner through the app using authorized medical practitioner access, record all uses through the app, and set, adjust, and power the heat source and vibration source 518, 528 in the mask 512, 522.

While preferred embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that any claims presented define the scope of the various embodiments and that methods and structures within the scope of these claims and their equivalents be covered thereby.

Embodiments described herein, therefore, are well adapted to carry out the objects and attain the ends and advantages mentioned, as well as others inherent therein. While a presently preferred embodiment has been given for purposes of disclosure, numerous changes exist in the details of procedures for accomplishing the desired results. These and other similar modifications will readily suggest themselves to those skilled in the art, and are intended to be encompassed within the spirit of the various embodiments disclosed herein and the scope of the appended claims.

Although the various embodiments have been described in detail, it should be understood that various changes, substitutions, and alterations can be made hereupon without departing from the principle and scope of the various embodiments. Accordingly, the scope of the various embodiments should be determined by the following claims and their appropriate legal equivalents.

The singular forms “a,” “an,” and “the” include plural referents, unless the context clearly dictates otherwise.

Optional or optionally means that the subsequently described event or circumstances can or may not occur. The description includes instances where the event or circumstance occurs and instances where it does not occur.

Ranges can be expressed herein as from about one particular value, and/or to about another particular value. When such a range is expressed, it is to be understood that another embodiment is from the one particular value and/or to the other particular value, along with all combinations within said range.

As used herein and in the appended claims, the words “comprise,” “has,” and “include” and all grammatical variations thereof are each intended to have an open, non-limiting meaning that does not exclude additional elements or steps.

As used herein, terms such as “first” and “second” are arbitrarily assigned and are merely intended to differentiate between two or more components of an apparatus. It is to be understood that the words “first” and “second” serve no other purpose and are not part of the name or description of the component, nor do they necessarily define a relative location or position of the component. Furthermore, it is to be understood that the mere use of the term “first” and “second” does not require that there be any “third” component, although that possibility is contemplated under the scope of the various embodiments.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The embodiments can suitably comprise, consist or consist essentially of the elements disclosed and can be practiced in the absence of an element not disclosed.

What is claimed is:

1. A cloud-based ophthalmic eyelid treatment monitoring system, the monitoring system comprising:
a moldable warming device, the moldable warming device comprising:
a heating disc;
a resonance frequency stimulation vibration generator (RFSVG);
a coupling device;
a mask, wherein the mask is configured to hold the heating disc, the RFSVG, and the coupling device for use in parallel utility; and
a sensor array, wherein the sensor array is configured to generate a data stream responsive to a vibration and heating profile of a user's individual eyelid and periorbital three-dimensional anatomy and surface topography;
a monitor, wherein the monitor is configured to receive the data stream from the sensor array and configured to transmit the data stream to a cloud server; and
the cloud server, wherein the cloud server is configured to process the data stream so as to determine tuning parameters of the vibration and heating profile of the user's individual eyelid and periorbital three-dimensional anatomy and surface topography, and configured to transmit the tuning parameters to the monitor,
wherein the monitor is configured to receive the tuning parameters from the cloud server, configured to display information related to the tuning parameters, and configured to transmit the tuning parameters to the moldable warming device, and
wherein the moldable warming device is configured to receive the tuning parameters from the monitor and configured to generate thermal and vibratory energy according to the tuning parameters.

2. The monitoring system of claim 1, wherein the moldable warming device is configured to provide an entire eyelid surface and periorbital structures with therapeutic warmth and tuned harmonic resonance frequency stimulation vibration to mobilize Meibum lipids and stimulate flow of the mobilized Meibum lipids from the user's Meibomian glands.

3. The monitoring system of claim 1, wherein the coupling device is configured to contact the user's eyelid skin for transferring thermal and vibratory energy.

4. The monitoring system of claim 1, wherein the sensor array comprises sensors selected from the group consisting of: temperature sensors, pressure sensors, moisture sensors, pH sensors, and combinations thereof.

5. The monitoring system of claim 1, wherein the moldable warming device further comprising:
a microprocessor; and
a transmitter,
wherein the data stream generated by the sensor array is analog,
wherein the microprocessor receives the data stream from the sensor array and converts the data stream into digital, and
wherein the transmitter receives the data stream from the microprocessor and transmits the data stream wirelessly.

6. The monitoring system of claim 5, wherein the monitor receives the data stream wirelessly transmitted from the transmitter.

7. The monitoring system of claim 1, wherein the monitor is configured to wirelessly transmit the data stream to the cloud server.

8. The monitoring system of claim 1, wherein the cloud server is configured to wirelessly transmit the tuning parameters to the monitor.

9. The monitoring system of claim 1, wherein the monitor is configured to wirelessly transmit the tuning parameters to the moldable warming device.

10. The monitoring system of claim 1, further comprising:
a cloud computing unit, wherein the cloud computing unit utilizes one selected from the group consisting of: artificial intelligence, big-data pattern recognition, and combinations thereof, to optimize the tuning parameters.

11. The monitoring system of claim 1, wherein the cloud server comprises a database of information selected from the group consisting of: treatment data, sensor data, meta-data, patient-provided data, and combinations thereof.

12. A cloud-based ophthalmic eyelid treatment monitoring system, the monitoring system comprising:
a cloud server, wherein the cloud server is configured to determine a first set of tuning parameters related to a heating profile of a user's individual eyelid and periorbital three-dimensional anatomy and surface topography, and is configured to transmit the first set of tuning parameters to an in-office monitor of an in-office system and an at-home monitor of an at-home system;
the in-office system, the in-office system comprising:
an in-office mask comprising a first heat source; and
the in-office monitor,
wherein the in-office monitor is configured to transmit the first set of tuning parameters to the in-office mask for the first heat source to generate thermal energy according to the first set of tuning parameters; and
the at-home system, the at-home system comprising:
an at-home mask comprising a second heat source; and
the at-home monitor,
wherein the at-home monitor is configured to transmit the first set of tuning parameters to the at-home mask for the second heat source to generate thermal energy according to the first set of tuning parameters.

13. The monitoring system of claim 12, wherein the cloud server utilizes one selected from the group consisting of: artificial intelligence, big-data pattern recognition, and combinations thereof, to optimize the first set of tuning parameters.

14. The monitoring system of claim 12, wherein the cloud server comprises a database of information selected from the group consisting of: treatment data, sensor data, meta-data, patient-provided data, and combinations thereof.

15. The monitoring system of claim 12, wherein the in-office mask further comprises at least one sensor, wherein the at least one sensor is configured to generate a data stream responsive to the heating profile of the user's individual eyelid and periorbital three-dimensional anatomy and surface topography, and wherein the in-office monitor is configured to receive the data stream from the at least one sensor and to transmit the data stream to the cloud server.

16. The monitoring system of claim 12, wherein the at-home mask further comprises at least one sensor, wherein the at least one sensor is configured to generate a data stream responsive to the heating profile of the user's individual eyelid and periorbital three-dimensional anatomy and surface topography, and wherein the at-home monitor is configured to receive the data stream from the at least one sensor and to transmit the data stream to the cloud server.

17. The monitoring system of claim 12, wherein the cloud server is configured to determine a second set of tuning parameters related to a vibration profile of the user's individual eyelid and periorbital three-dimensional anatomy and surface topography, and is configured to transmit the second set of tuning parameters to the in-office monitor of the in-office system and the at-home monitor of the at-home system.

18. The monitoring system of claim 17, wherein the cloud server utilizes one selected from the group consisting of: artificial intelligence, big-data pattern recognition, and combinations thereof, to optimize the second set of tuning parameters.

19. The monitoring system of claim 17, wherein the in-office mask further comprises a resonance frequency stimulation vibration generator (RFSVG), wherein the in-office monitor is configured to transmit the second set of tuning parameters to the in-office mask for the RFSVG to generate vibrational energy according to the second set of tuning parameters.

20. The monitoring system of claim 17, wherein the at-home mask further comprises a resonance frequency stimulation vibration generator (RFSVG), wherein the at-home monitor is configured to transmit the second set of tuning parameters to the at-home mask for the RFSVG to generate vibrational energy according to the second set of tuning parameters.

21. The monitoring system of claim 17, wherein the in-office mask further comprises at least one sensor, wherein the at least one sensor is configured to generate a data stream responsive to the vibration profile of the user's individual eyelid and periorbital three-dimensional anatomy and surface topography, and wherein the in-office monitor is configured to receive the data stream from the at least one sensor and to transmit the data stream to the cloud server.

22. The monitoring system of claim 17, wherein the at-home mask further comprises at least one sensor, wherein the at least one sensor is configured to generate a data stream responsive to the vibration profile of the user's individual eyelid and periorbital three-dimensional anatomy and surface topography, and wherein the at-home monitor is configured to receive the data stream from the at least one sensor and to transmit the data stream to the cloud server.

23. A cloud-based method for monitoring ophthalmic eyelid treatment, the method comprising the steps of:
applying a moldable warming device to a user's individual eyelid and periorbital three-dimensional anatomy and surface topography, the moldable warming device comprising:
a heating disc;
a resonance frequency stimulation vibration generator (RFSVG);
a coupling device;
a mask, wherein the mask is configured to hold the heating disc, the RFSVG, and the coupling device for use in parallel utility; and
a sensor array;
generating, at the sensor array, a data stream responsive to a vibration and heating profile of the user's individual eyelid and periorbital three-dimensional anatomy and surface topography;
communicating the data stream from the sensor array to a monitor;
transmitting the data stream from the monitor to a cloud server;
processing, at the cloud server, the data stream so as to determine tuning parameters of the vibration and heating profile of the user's individual patient eyelid and periorbital three-dimensional anatomy and surface topography;
transmitting the tuning parameters from the cloud server to the monitor;
displaying, on the monitor, information related to the tuning parameters;
communicating the tuning parameters from the monitor to the moldable warming device; and
generating, at the moldable warming device, thermal and vibratory energy according to the tuning parameters.

24. The method of claim 23, wherein the moldable warming device is configured to provide an entire eyelid surface and periorbital structures with therapeutic warmth and tuned harmonic resonance frequency stimulation vibration to mobilize Meibum lipids and stimulate flow of the mobilized Meibum lipids from the user's Meibomian glands.

25. The method of claim 23, further comprising the step of:
converting the data stream from analog to digital.

26. The method of claim 23, wherein the data stream is communicated wirelessly in the communicating the data stream step.

27. The method of claim 23, wherein the data stream is transmitted wirelessly in the transmitting the data stream step.

28. The method of claim 23, wherein the tuning parameters are transmitted wirelessly in the transmitting the tuning parameters step.

29. The method of claim 23, wherein the tuning parameters are communicated wirelessly in the communicating the tuning parameters step.

30. A cloud-based method for monitoring ophthalmic eyelid treatment, the method comprising the steps of:
determining, at a cloud server, a first set of tuning parameters related to a heating profile of a user's individual eyelid and periorbital three-dimensional anatomy and surface topography;
transmitting the first set of tuning parameters from the cloud server to at least one monitor;
communicating the first set of tuning parameters from the at least one monitor to at least one mask, wherein the at least one mask includes a heat source; and
generating, at the at least one mask, thermal energy according to the first set of tuning parameters.

31. The method of claim 30, further comprising the step of:
displaying, on the at least one monitor, information related to the first set of tuning parameters.

32. The method of claim 30, further comprising the steps of:
generating a data stream, at at least one sensor, responsive to the heating profile of a user's individual eyelid and periorbital three-dimensional anatomy and surface topography;
communicating the data stream from the at least one sensor to the at least one monitor; and
transmitting the data stream from the at least one monitor to the cloud server.

33. The method of claim 30, further comprising the steps of:
determining, at the cloud server, a second set of tuning parameters related to a vibration profile of the user's individual eyelid and periorbital three-dimensional anatomy and surface topography; and
transmitting the second set of tuning parameters from the cloud server to the at least one monitor.

34. The method of claim 33, further comprising the step of:
displaying, on the at least one monitor, information related to the second set of tuning parameters.

35. The method of claim 33, further comprising the steps of:
communicating the second set of tuning parameters from the at least one monitor to the at least one mask, wherein the at least one mask includes a resonance frequency stimulation vibration generator (RFSVG); and
generating, at the at least one mask, vibrational energy according to the second set of tuning parameters.

36. The method of claim 33, further comprising the steps of:
generating a data stream, at at least one sensor, responsive to the vibration profile of the user's individual eyelid and periorbital three-dimensional anatomy and surface topography;
communicating the data stream from the at least one sensor to the at least one monitor; and
transmitting the data stream from the at least one monitor to the cloud server.
